(19) United States
(12) Patent Application Publication
(10) Pub. No.: US 2024/0249831 A1
(43) Pub. Date: Jul. 25, 2024

(54) PLATFORM FOR DETERMINING A COMPETENCY SCORE

(71) Applicant: Firefly Lab, LLC, Wilmington, DE (US)

(72) Inventors: Jonathan Lee Jesneck, Lake Oswego, OR (US); Ruchi Mrugesh Thanawala, Lake Oswego, OR (US)

(73) Assignee: Firefly Lab, LLC, Wilmington, DE (US)

(21) Appl. No.: 18/628,457

(22) Filed: Apr. 5, 2024

Related U.S. Application Data
(63) Continuation of application No. PCT/US2022/046131, filed on Oct. 8, 2022.
(60) Provisional application No. 63/254,040, filed on Oct. 8, 2021.

(51) Int. Cl. G16H 40/20

(52) U.S. Cl. CPC G16H 40/20

(57) ABSTRACT

The invention provides platforms and methods for determining competency scores and predicting outcomes for healthcare professionals. The platforms and methods utilize performance evaluations obtained from evaluator healthcare professionals for a target healthcare professional and matched peer group, and can be used for evaluating current performance and predicting future performance.

### CROSS-REFERENCE TO RELATED APPLICATIONS

This continuation application claims the benefit of and priority to International Patent Application No. PCT/US2022/046131, filed on Oct. 8, 2022, which claims the benefit of and priority to U.S. Provisional Application No. 63/254,040, filed on Oct. 8, 2021.

### FIELD OF THE INVENTION

The present invention provides a platform for systems and methods for determining competency and risk scores and predicting outcomes for healthcare professionals. These systems and methods utilize performance evaluations from a group of evaluator healthcare professionals for a target healthcare professional and also a matched peer group of the target healthcare professional, for the performance of one or more selected medical procedures. These systems and methods are useful not only for determining a current competency and risk score, but also for predicting scores for future medical tasks.

### BACKGROUND OF THE INVENTION

Aggregation of clinical and surgical education data is essential for individual trainees, residency programs, education policy makers, and quality-improvement initiatives. However, such data are typically siloed, difficult to access, and burdensome to integrate with other information. Such information is useful to also assess the overall quality and performance of a hospital, university teaching facility, or other such institution, particularly in view of outcomes and associated insurance reimbursement considerations.

Most medical, clinical, educational, and research initiatives can be greatly facilitated by convenient access to relevant, high-quality data. However, medical and research data are notoriously difficult to access, aggregate, and standardize, especially in a HIPAA-compliant manner. Additionally, hospital staff time constraints and data-entry burnout can easily stymie any new data collection efforts. These issues are further compounded by the complexity of sharing data across institutions.

For example, the American Board of Surgery expects surgical residents to be proficient, safe, and autonomous across 132 “Core” surgical procedures in order to graduate and become practicing surgeons. For surgical educators, it can be a daunting task to solicit and assimilate performance feedback across a program's residents, especially in a timely, comprehensive, and quantitative manner. The situation is similar across other fields of medicine, and not only for surgical and other residents, but also for interns, medical students, nurses, technicians, and other healthcare professionals.

Doctors that are completing their interning and medical residency requirements undergo rigorous and demanding training to become proficient in their chosen field of specialization. A general surgery residency in the United States is currently five years. Specialization in a surgical specialty will add on additional years of training. For example, to specialize in thoracic surgery requires an additional two years of residency. Despite the rigors of such training, some residents may not be receiving the hands-on surgical experiences, training, mentoring, feedback, and any interventional or remedial actions in a timely manner. Part of the reason for this lack of training and feedback is that the procedures for inputting and documenting resident performance information is time consuming and inefficient, which can result in the information not being timely or adequately documented. This situation with information that is not timely or adequately input can lead to residents not knowing where they stand in terms of their training requirements and their performance of medical procedures. Also, performance feedback information that is not timely or adequately input can be detrimental to the learning and performance of a resident. Therefore, the present system for training surgical residents is not fully designed to track and optimize resident performance, which can result in a proportion of residents not being able to successfully complete their residency. Although these shortcomings of residency training are described with a focus on surgical residencies, these shortcomings are common to residencies in other areas of medicine. Additionally, these shortcomings are also found across other areas of medical training including internships, nurse training programs, physician assistant programs, and other professional areas for technicians such as for inhalation therapy and for the operation of specialized diagnostic equipment.

With the current pedagogical model for physician training, objective and timely performance evaluation information about how well trained and proficient a doctor, such as a surgeon, is with performing a specific medical procedure can be lacking. For surgical training in residency, the Accreditation Council for Graduate Medical Education (ACGME) does not attempt to measure when a resident is “ready” to graduate, but instead has a minimum number of cases that the resident is required to perform to meet the accreditation standards. It has been published that most surgery residents report that they do not feel adequately prepared to practice when they graduate from their residency, and that each resident needs a different number of cases in order to become proficient in performing a procedure. See, Yeo, Heather, et al. “Attitudes, training experiences, and professional expectations of US general surgery residents: a national survey.” Jama 302.12 (2009): 1301-1308; Stride, Herbert P., et al. “Relationship of procedural numbers with meaningful procedural autonomy in general surgery residents.” Surgery 163.3 (2018): 488-494; Abbott, Kenneth L., et al. “Number of Operative Performance Ratings Needed to Reliably Assess the Difficulty of Surgical Procedures.” Journal of surgical education 76.6 (2019): e189-e192; and Williams, Reed G., et al. “How many observations are needed to assess a surgical trainee's state of operative competency?.” Annals of surgery 269.2 (2019): 377-382.

Also, educational quality varies across institutions. Some institutions allow residents to dig in and get hands-on experience doing procedures early in their career, whereas other institutions require residents for the first year or two to only stand to the side of the operating table to observe the procedure over a surgeon's shoulder. Further complicating physician training and competency evaluation is that the standard way for assessing physician expertise and competency is from the physician's patient outcomes over a significant period of time—this can often be years. It is well known that outcomes depend on other factors, such as the underlying health of the patient, level and quality of post-operative care, insurance reimbursement, etc. Because of these other factors, it is difficult to determine which portion of the outcome is directly attributable to a single doctor.

Furthermore, there is a need to assess the safety or risk associated with a particular healthcare professional, or groups or a team of healthcare professionals, performing a particular medical procedure, or even with a clinic or hospital in performing a particular type of medical procedure. This risk may be quantified in a risk score that can be used to predict the probability of a clinical event achieving a relevant metric, such as patient outcome. The risk score could be used to make informed staffing and hiring decisions at a hospital or clinic, for determining when a patient should stay in-house or be transferred to another facility for medical care, and to align financial incentives, such relative value units (RVUs), to optimize hospital/clinic efficiency, maximize revenue, and reduce risk. Another contemplated application is in the optimization of insurance policies and insurance rates. A further contemplated application is in the maintenance of certification and continuing education for medical providers and instructors.

Additionally, competency and risk scores may also be calculated and used in applications not directly related to patient safety, such as, for example, to assess nursing students who are learning to administer pain control protocols.

To address the forgoing needs and shortcomings, we have therefore developed a learning curve model for building a method and data management system for the tracking and optimization of medical clinical performance for healthcare professionals, and in particular for medical residents such as surgical residents. This model is the basis of our platform for medical scoring and profiling. The methods and systems of the present invention intelligently aggregate and anonymize large volumes of data, and can optimize data-entry workflows to make new data collection efforts feasible, thereby facilitating training and optimizing performance for healthcare professionals. The present invention provides a platform for systems and methods for determining competency scores and predicting outcomes for healthcare professionals. Furthermore, the information used to develop these models is input and organized to facilitate the operation and efficiency of the computer and data storage systems.

Prior to the platform of the present invention, it was difficult, if not impossible to evaluate the huge amount of data that this platform integrates to provide meaningful and useful performance assessments for healthcare professional on a reasonably short time scale.

The systems and methods of the present invention provide the following features and advantages over the solutions currently available. The invention uses computational models, such as statistical models, deep learning models, and machine learning models, to infer a medical practitioner's ability and to project the learning and ability in the future as the practitioner gains more clinical experience. The invention uses statistical models to account for confounders, such as the evaluator's “hawk vs dove” rater bias and the clinical case complexity. The invention is flexible and can use many types of evaluations, rather than requiring a defined evaluation type up front. The invention can be used to create scores and profiles for medical expertise and autonomy, i.e., competency, which can be used in a variety of ways. Also, the platform and evaluations are not dependent on predefined job goals. Additionally, the following features describe the methods and systems of the present invention.

The present invention also automates medical chores such as surgical data chores. The features provide for: scheduling and quickly assigning cases; automated case logging into the databases for the ACGME, the American College of Surgeons (ACS), etc.; quick evaluations for early, useful doctor feedback; live analytics for improvement tracking; and curated educational content to facilitate case preparation. These foregoing features result in saved time, particularly for case logging alone.

### SUMMARY OF THE INVENTION

The present invention provides a platform that uses medical and educational activity data, such as clinical schedules, training exercises, and educational activities, to track professional medical tasks and assess their complexity. By pairing these tasks with performance evaluations for healthcare practitioners, the platform uses computational models for each type of medical task to construct learning curves and calculates the practitioner's individual competency score and outcome risk score. Future scores are predicted using learning curves and future scheduled activities.

The present invention relates to a platform, such as a web-based platform, to track resident operative assignments and to link embedded evaluation instruments to procedure type. The platform of the present invention provides an improvement upon conventional methods and systems for tracking and evaluating resident performance and provides an important training tool for advancing resident knowledge and skill development.

Additionally, these methods and systems are contemplated as being applicable across other areas of the medical profession and include internships, nurse training programs, physician assistant programs, and other professional areas for technicians such as for inhalation therapy and for the operation of specialized diagnostic equipment.

The present invention includes, among other things, the following embodiments.

The present invention provides a data management platform for determining a competency score for a target healthcare professional, the platform comprising: a computer, a server or data storage system, a user interface, a non-transitory computer-readable medium storing computer program instructions, software for analyzing input data and providing an output, and a data array, wherein the platform is configured to perform steps comprising:
acquiring clinical schedules indicating clinical procedures to be performed;
listing component tasks and required skills for each procedure;
assessing task complexity; collecting performance evaluations for a target healthcare professional and a matched peer group of the target healthcare professional, for the performance of one or more selected procedures, each procedure having one or more tasks and an assigned clinical complexity value for the procedure and the one or more tasks thereof;
compiling the evaluations versus predetermined standards for the successful completion of each task and one or more steps thereof to provide performance parameters;
performing a computation to produce learning curves from the performance parameters for the target healthcare professional and the matched peer group of the target healthcare professional, wherein the computation is selected from the group consisting of statistical modeling, deep learning modeling, and machine learning modeling; from the learning curves for the target healthcare professional, calculating a competency score for the target healthcare professional for the procedure and each task thereof; and
comparing the learning curves and skill levels for the procedure and each task thereof for the target healthcare professional to that of the matched peer group of the target healthcare professional to determine a competency score for the target healthcare professional.

The present invention provides a platform wherein the computation is deep learning modeling.

The present invention provides a platform wherein the deep learning modeling is a learning curve modeling.

The present invention provides a platform wherein the learning curve modeling comprises the step of performing a statistical sampling method calculation to produce one or more learning curves for the target healthcare professional and the matched peer group of the target healthcare professional.

The present invention provides a platform wherein the computation is statistical modeling.

The present invention provides a platform wherein the computation is machine learning modeling.

The present invention provides a platform wherein the healthcare professional is selected from the group consisting of medical students, interns, residents, fellows, doctors, physician assistants, nurses, nurses' aides, and medical technicians.

The present invention provides a platform involving a teaching situation involving an evaluator healthcare professional and a target healthcare professional.

The present invention provides a platform wherein the user interface is selected from the group consisting of a graphical user interface, a command-line interface, and a menu driven interface.

The present invention provides a platform wherein the user interface is a graphical user interface.

The present invention provides a platform wherein the graphical user interface is configured to augment a clinical schedule with case-based actions; the graphical user interface comprising:
a first element showing a staff assignment for a clinical encounter; and
a second element juxtaposed to the first element and showing a button, a tag, a status label, or an actionable link for an encounter-related activity, such as case logging, performance evaluation, data quality control, and accessing medical educational content.

The present invention provides a platform wherein the performance evaluations are provided manually.

The present invention provides a platform wherein the performance evaluations are provided by artificial intelligence.

The present invention provides a platform that is a web-based platform.

The present invention provides a platform wherein the platform is embedded in a hospital data system.

The present invention provides a platform wherein the hospital data system is an electronic health record system.

The present invention provides a platform that is Health Insurance Portability and Accountability Act compliant.

The present invention provides a platform configured to comprise a step of determining a risk score, wherein the risk score indicates a probability of a clinical event achieving a predetermined patient outcome.

The present invention provides a method for determining a competency score for a target healthcare professional comprising the following steps:
acquiring clinical schedules indicating clinical procedures to be performed;
listing component tasks and required skills for each procedure;
assessing task complexity;
collecting performance evaluations for a target healthcare professional and a matched peer group of the target healthcare professional, for the performance of one or more selected procedures, each procedure having one or more tasks and an assigned clinical complexity value for the procedure and the one or more tasks thereof;
compiling the evaluations versus predetermined standards for the successful completion of each task and one or more steps thereof to provide performance parameters;
performing a computation to produce learning curves from the performance parameters for the target healthcare professional and the matched peer group of the target healthcare professional, wherein the computation is selected from the group consisting of statistical modeling, deep learning modeling, and machine learning modeling;
from the learning curves for the target healthcare professional, calculating a competency score for the target healthcare professional for the procedure and each task thereof; and
comparing the learning curves and skill levels for the procedure and each task thereof for the target healthcare professional to that of the matched peer group of the target healthcare professional to determine a competency score for the target healthcare professional.

The present invention provides a method wherein the computation is deep learning modeling.

The present invention provides a method wherein the deep learning modeling is a learning curve modeling.

The present invention provides a method wherein the learning curve modeling comprises the step of performing a statistical sampling method calculation to produce one or more learning curves for the target healthcare professional and the matched peer group of the target healthcare professional.

The present invention provides a method wherein the healthcare professional is selected from the group consisting of medical students, interns, residents, fellows, doctors, physician assistants, nurses, nurses' aides, and medical technicians.

The present invention provides a method involving a teaching situation involving an evaluator healthcare professional and a target healthcare professional.

The present invention provides a method wherein the user interface is selected from the group consisting of a graphical user interface, a command-line interface, and a menu driven interface.

The present invention provides a method wherein the user interface is a graphical user interface.

The present invention provides a method wherein the graphical user interface is configured to augment a clinical schedule with case-based actions; the graphical user interface comprising:
a first element showing a staff assignment for a clinical encounter; and
a second element juxtaposed to the first element and showing a button, a tag, a status label, or an actionable link for an encounter-related activity, such as case logging, performance evaluation, data quality control, and accessing medical educational content.

The present invention provides a method wherein the performance evaluations are provided manually.

The present invention provides a method wherein the performance evaluations are provided by artificial intelligence.

The present invention provides a method that utilizes a web-based platform.

The present invention provides a method wherein the platform is embedded in a hospital data system.

The present invention provides a method wherein the hospital data system is an electronic health record system.

The present invention provides a method that is Health Insurance Portability and Accountability Act compliant.

The present invention provides a method comprising determining a risk score, wherein the risk score indicates a probability of a clinical event achieving a predetermined patient outcome.

The present invention provides a method wherein the risk score is calculated for an individual practitioner to perform a specific procedure.

The present invention provides a method comprising determining a multi-task aggregate competency score based on individual task scores for an overall procedure.

The methods and systems disclosed herein often are used to process huge amounts of various types of data in near real time, and it goes without saying that such large-scale data operations are not something that could be performed solely with the human mind or by pen and paper.

These and other aspects of the present invention will become apparent from the disclosure herein.

### Definitions

The terms “practitioner” and “medical professional”, as used herein, include such roles as a medical student, resident, fellow, technician, attending, nurse, physician assistant, etc.

The term “procedure”, as used herein, refers to any action, decision, responsibility, etc. that is relevant for a medical practitioner/training to perform in the course of her/his job or training. A procedure will comprise one or more tasks as defined herein. Exemplary procedures may include: determining a medical diagnosis, performing a surgical procedure, performing medical imaging of a patient, interpreting medical imaging, setting up medical equipment, using a piece of medical equipment, prescribing medication, administering medication, administering physical therapy, administering psychological therapy or counseling, harvesting/collecting biological sample, testing a biological sample, performing a simulation exercise for training, etc.

The term “task”, as used herein, refers to a component or step of a procedure. A task is usually a small, discrete action and a procedure typically consists of multiple tasks. Exemplary tasks may include: tying a knot while suturing a wound closed, positioning the patient in a CT imaging device, listening to the lungs while doing a physical exam on a patient, placing a cuff on an arm to measure blood pressure, counting the pulses when measuring heart rate, entering the dosage value into the radiation software when preparing to administer radiation therapy, etc. All non-trivial medical procedures can be described as a series (or branching map with decision points) of tasks.

The term “score”, as used herein, refers to a measure of a quantity, especially one relevant to performing a procedure or task, or to the outcome thereof. A score may be quantified and reported in various formats, including a probability distribution, a value range, a single numerical value, a ranking, a value in an ordinal value scale, etc.

The terms “competency” and “skill”, and “competency score” and “skill score”, are used synonymously herein, and refer to a measure indicating a medical practitioner's or trainee's knowledge and ability to perform a procedure or task. The terms “entrustability”, “autonomy”, and “expertise” are yet additional synonyms. A competency score may be calculated for a single individual, a team, a department, entire hospital, etc. Moreover, a competency score may be calculated for a portion of a task, a whole task, a procedure, a set of procedures, etc.

The term “outcome”, as used herein, refers to an end result of a procedure or task. Some examples of outcomes include: the successful removal of a cancer with the patient in a stable condition; a blurry MRI image due to suboptimal usage of the MRI machine; a reduced pain level in a patient due to application of pain medication; a correct diagnosis of a heart attack; no apparent change in depression levels after a psychological treatment; a patient death due to bleeding from gunshot wounds during emergency surgery; an incorrect diagnosis of an ear infection, etc.

A task may result in any one of a variety of outcomes, depending on initial patient conditions, medical team competency levels, available medical equipment and personnel, treatment decisions during the task, and random chance.

The term “acceptability”, as used herein, refers to the overall assessment of satisfaction by a medical professional or organization for a task outcome. The satisfaction may be based on various factors, such as, for example, patient health and safety, patient peace of mind, financial cost, adherence to or deviation from standard care practice, time and resource efficiency, and patient quality-of-life after the task is performed, etc.

The terms “risk” and “safety”, and “risk score” and “safety score”, are used synonymously herein, and refer to the probability that a procedure or task will result in an outcome below a minimum acceptability threshold. A risk score may be calculated based on one or multiple acceptability factors. In certain embodiments, a risk score may be calculated to predict the risk for a cardiac surgery team to fail at replacing a mitral valve; the risk for a group of obstetricians to incur a major lawsuit within a year period; the risk for an x-ray technician to operate imaging equipment with such that less than 95% of images are in focus with adequate contrast to be readable by radiologists; the risk for a nursing group to fail to identify signs of post-surgical site infection and apply antibiotic medication for more than 1% of site infections; the risk for a phlebotomist to severely injure a vein while drawing blood samples; and the risk of an emergency department nurse failing to identify more than 2 strokes during a year period.

### BRIEF DESCRIPTION OF THE DRAWINGS

FIG. 1 is a flow diagram for the determination of a competency score and a related outcomes risk score for a medical practitioner.

FIG. 2 is a flow diagram of an embodiment of the preprocessing and curation of data that is input into the flow illustrated in FIG. 1.

FIG. 3 is a representation of a practitioner's, i.e., a healthcare professional's, competency score per task in a particular procedure. The competency score is computed from the practitioner's learning curve associated with the task.

FIG. 4 is a task process model, indicating examples of various probabilities of a medical practitioner or team transitioning between states and the likely outcomes and acceptabilities thereof.

FIG. 5A is a pie-chart representation of probabilities and acceptabilities of various task outcomes. Medical outcome probabilities are calculated from the complexity of the medical task and the practitioner's current skill (competency) level for that task. FIG. 5B is a representation of the risk score distribution, based on the task outcome probabilities and associated outcome acceptabilities of FIG. 5A. The risk score distribution is calculated based on the occurrence probabilities and medical consequences of the task/procedure outcomes (e.g., effects on patient health) and defines the practitioner's ability to perform a given task/procedure independently and safely. This score can be used for staffing decisions for medical procedures and for guiding the training of medical professionals.

FIG. 6A shows example competency scores for a particular task performed by practitioners A-E who are members of a team; FIG. 6B shows an example composite competency score of the entire team for that particular task.

FIG. 7A is a pie-chart representation of a distribution of outcome probabilities for multiple component tasks of a procedure, and also shows a related competency score (either for an individual practitioner or for a group of practitioners) for the tasks. FIG. 7B displays an exemplary aggregate competency distribution for the tasks.

FIG. 8 is a flow diagram of an embodiment of the procedure process mapping performed by the method disclosed herein.

FIG. 9 is a flow diagram of an embodiment of the model training and validation steps performed by the method disclosed herein.

FIG. 10 is an illustration of how the platform of the present invention connects multiple systems and users.

FIG. 11 is a diagram for the scoring and profiling system for medical providers and learners, showing the input data and output profiles.

FIG. 12 is an illustration of a user interface, such as a graphical user interface (GUI), where the case logging and evaluations are integrated into the medical professional's schedule.

FIG. 13 is a diagram showing the data flow and processing for the code matching engine to identify and rank order smart suggestions and smart search for appropriate medical codes for medical activities.

FIG. 14 is a diagram showing the data flow and processing for the content matching engine to identify and rank order smart suggestions and smart search for targeted educational material and exercises for a medical practitioner.

FIG. 15 is a diagram showing an embodiment of the statistical learning curve model as disclosed herein, to infer the expertise or autonomy level of a medical professional for a procedure.

FIG. 16 is a diagram showing the modeling of resident (practitioner) learning and autonomy.

FIG. 17 is a plot showing exemplary learning curves for individual residents (practitioners) for laparoscopic cases. This figure illustrates the modeling process where what is plotted is the most likely (maximum a posteriori estimate) learning curve for each surgery resident in a small group of residents that were having difficulty learning laparoscopic procedures. Individual lines show the learning curves of each individual resident. The horizontal axis is the number of procedures that the resident performed over time, and the vertical axis is the autonomy score (the higher, the more independent the resident). The dots show some individual evaluations received over time.

FIG. 18 is a plot showing posterior samples of exemplary learning curves for the residents, both before the teaching intervention and after the intervention of the method disclosed herein.

FIG. 19 shows a vertical slice cross-section of the bands at the far right of FIG. 17. This data relates to predictive distributions for maximum resident autonomy. These data show that the intervention worked and made those residents more independent in the operating room.

FIG. 20 is a bar graph illustrating laparoscopic procedural autonomy for an intervention, i.e. additional teaching support, and non-intervention group of residents.

FIG. 21 is a diagram showing the evaluation lifecycle as deployed by the system and methods disclosed herein.

FIG. 22 is a plot showing exemplary learning curves for a group of residents as a function of cases (or procedures) performed. The y-axis shows the level of autonomy rating, from lowest to highest: attending surgeon performed; steered; prompted; back-up, and auto as described further in Table 2. The x-axis shows the number of cases (procedures) performed by the resident (practitioner).

FIG. 23 shows a plot of exemplary resident performance across evaluations as Operative Performance Rating System (OPRS) overall score versus O-score overall score. The Operative Performance Rating System (OPRS) is a set of procedure-specific rating instruments and recommended methods for observing and judging single performances of nine operative procedures frequently performed by general surgeons and by general surgery residents-in-training. The O-score is the Ottawa Surgical Competency Operating Room Evaluation (O-SCORE) and is a 9-item surgical evaluation tool designed to assess technical competence in surgical trainees using behavioral anchors.

FIGS. 24A and 24B are plots showing how the methods and systems of the present invention are useful for predicting case (procedure) volume. FIG. 24A shows total resident case (procedure) volume while FIG. 24B shows the case (procedure) volume for an individual resident.

### DETAILED DESCRIPTION OF THE INVENTION

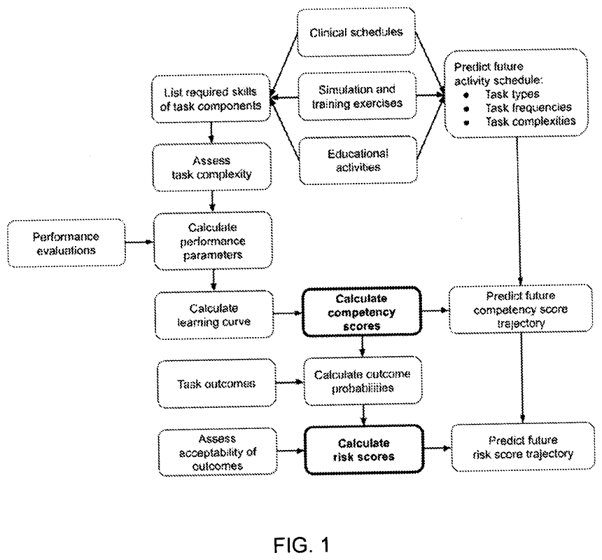

FIG. 1 is an exemplary flow diagram of the steps for calculating competency and risk scores. In accordance with the flow shown in FIG. 1, the first step in the data processing flow is to gather the relevant experience of medical professionals, such as historical clinical schedules and activity logs, simulation and training exercises, and educational activities. Clinical schedules and activity logs may include the set of clinical, administrative, and operative schedules from the hospital or clinic, typically recorded in their electronic health record (EHR) system; simulation and training exercises may be a set of training exercises that medical and surgical training programs provide for their trainees; and educational activities may include any available relevant educational activity, such as study group sessions, lectures, courses, testing, etc. This data may come from hospital records or may be entered manually by an individual resident or program manager. Data obtained from hospital records, for example, may need to be preprocessed to standardize its format, eliminate inaccuracies, or supplement missing information.

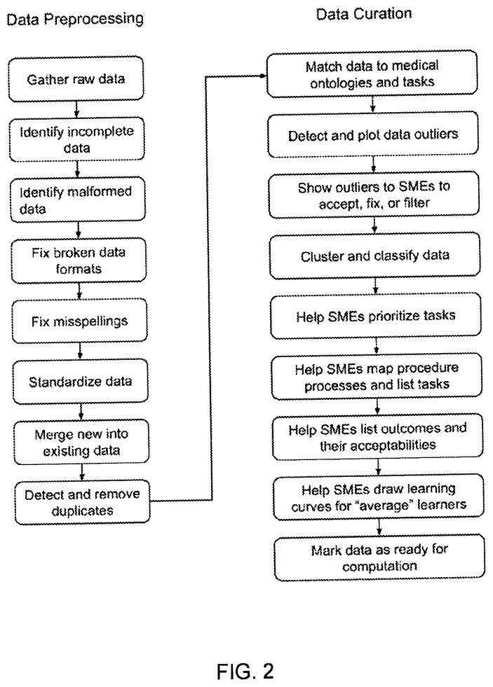

FIG. 2 shows a detailed example of steps that may be performed to preprocess and curate the data. Raw data is checked for completeness and irregularities. Improperly formatted data is repaired. Spellings of terminologies and procedures are corrected if necessary. Data is standardized and any new data is merged into a database of existing data. Duplicate entries are removed. The data is then curated by identifying outliers, consulting with subject matter experts (SMEs) to accept, fix, or filter any such outlying data, and, at least, by listing and prioritizing tasks to be performed as part of an overall medical procedure. Each medical procedure to be performed consists of one or more smaller steps, referred to herein as tasks. The various steps that may be involved in the data preprocessing and data curation of FIG. 2 are summarized below.

### Data Preprocessing:

Gather raw data: Collect raw data from various sources, including the hospital electronic health record (EHR) system, individual medical personnel and their support staff, medical educational teams, medical and surgical simulation centers.
Identify incomplete data: For each input data record, identify missing data fields, and determine whether the record has enough partial data to be useful.
Identify malformed data: Identify malformed data, such as incorrect data field types, invalid dates, invalid numerical values, etc.
Fix broken data formats: Extract accessible data from the broken format and fix the format. Corrupted file formats commonly include PDF, CSV, xlsx, and docx formats.
Fix misspellings: Scan for and automatically fix common misspellings of medical terms.
Standardize data: Translate data from any hospital site-specific formatting into standardized Firefly data formatting. This translation commonly includes handling synonyms and abbreviations of medical terms, date formats, and codifying any free-text that had been manually entered into external data systems.
Merge new into existing data: Identify any incoming data that matches to existing data in the Firefly database, and update any existing data records. Insert any new data that is not yet in the Firefly database.
Detect and remove duplicates: Identify duplicate records by any external identifiers, and by using multiple data fields, such as procedure date, room, and start time. Mark any duplicate records and delete them from the Firefly database.

### Data Curation:

Match data to medical ontologies and tasks: Match all practitioner activities and procedures to medical ontology systems. For example, we can match a Firefly surgical procedure to the corresponding surgical procedure in the ontology system, and assign the ontology record ID to the Firefly procedure.
Detect and plot data outliers: Identify data outliers, such as strange date values, and unusually high or low numerical values. Create plots of the data with any outliers.
Show outliers to SMEs to accept, fix, or filter: Plot the corresponding data for a data curation team and subject matter experts (SMEs), and highlight the outliers. Let the curators and SME review the data trends and outliers, and either accept the outliers flag them for exclusion from consideration when creating statistical and machine learning models.
Cluster and classify data: Cluster the data values, for inclusion into groups for computation. For example, procedure names can be grouped by procedure type, so that all the synonyms of the procedure across various hospitals are marked as belonging to the same procedure type. Numerical values are also clustered. For the evaluation performance ratings of trainees are clustered, to define matched peer groups for learners across institutions.
Help SMEs prioritize tasks: Medical educators can review and search through hundreds of medical procedures and tasks, and indicate which ones are especially important for training programs. Often trainees must achieve specific minimum requirements, such as a minimum number of various procedures, in order to graduate from their training program. The local educators may also prioritize procedures and tasks according to their local educational initiatives.
Help SMEs map procedure processes and list tasks: The SMEs can use the Firefly procedure mapping tool to draw the tasks and paths and transition probabilities. (This curation step is described in detail in the document “Details for process mapping flow diagram.docx”).
Help SMEs list outcomes and their acceptabilities: The SMEs can use the Firefly procedure mapping tool to list the procedure outcomes and mark the acceptability level of each outcome. (This is also described in “Details for process mapping flow diagram.docx”.)
Help SMEs draw learning curves for “average” learners: The SMEs can use a Firefly learning curve tool to draw the curve shape, in order to describe the learning process across most of their trainees. For example, in the curve parameters, the SME can define the learning rate and competency level over time, as the trainee gains more knowledge and experience in the procedure.
Mark data as ready for computation: Now that all the data preprocessing and curation are completed, the data is clean, standardized, and ready for computation (e.g. learning curve modeling, competency score modeling, and risk score modeling).

Returning to FIG. 1, the platform then lists required skills associated with the task or tasks. In this step, the process flow looks up the skills required to perform the task successfully. These required skills have been determined by subject matter experts (SMEs). Next, based on the number and difficulty level of the required skills, the platform computes and assesses task complexity. For example, a procedure for a laparoscopic cholecystectomy (i.e., a minimally invasive approach to removing a sick gallbladder) may include the component tasks of port placement, patient positioning, removing adhesions, dividing the cystic artery, etc., and require a skill level (competency score) of at least 0.75 on a 0-1 scale. Next, the platform calculates performance parameters based on a set of performance evaluations for the medical trainees and practitioners. The performance evaluations may include manual evaluations from medical professionals, augmented evaluations where a machine learning system facilitates a medical professional to complete the evaluation, or evaluations from autonomous machine learning systems. (The inventor's Firefly™ platform facilitates these evaluations by providing “smart” evaluations that are specific to the learner and the medical procedure and sends the evaluations to the teaching faculty soon after the procedure is competed.) From the evaluations, the platform extracts and organizes the performance ratings. A learning curve is then calculated by fitting a learning curve to the activity data and evaluation data and calculating the parameters of the learning curve model. Next, competency scores are calculated. In this step, for each task, the platform uses the learning curve to calculate the ability of the medical practitioner/team to perform the particular task. The score that is calculated corresponds to the learning curve value at the practitioner's current level of experience and performance for that particular task. Then, based on task complexities, practitioner/team competency scores, and a list of possible outcomes for the task(s), the platform calculates outcome probabilities, which indicate the probability of arriving at each task outcome at the conclusion of the procedure. Note that the list of possible outcomes are often entered by SMEs across tasks in their areas of expertise. The platform then acquires the acceptabilities of various outcomes, and calculates risk scores from the outcome probabilities and the acceptabilities. An acceptability indicates the acceptability of a particular outcome, and has been previously determined by SMEs. The calculated risk score indicates the probability the final outcome will be below a minimum acceptable level.

When the future clinical schedule is not available from the Electronic Health Record (EHR) system, the platform uses previous activity patterns to predict the likely types and frequencies of procedures for each practitioner. The learning curve predicts how competency levels will change as the practitioner gains more experience with a procedure. By combining the learning curve with the future clinical schedule (or if the future schedule is not available, a prediction of the procedure types and frequencies), the platform is able to predict future competency scores. Moreover, the platform may also predict the future risk score trajectory by using the predicted competency levels and the future clinical schedule (or predicted schedule) in order to predict the future risk scores.

The competency score and outcome risk scores have several useful applications, including aiding a medical service with its staffing decisions, to better ensure that adequately trained and skilled professionals are performing each procedure competently and safely. In addition to calculating these scores, the platform can use the learning curves and future scheduled activities to predict the future trajectory of these scores. The learning curves and future predicted values are useful for professional development, to help provide each medical practitioner with the appropriate training. Furthermore there is an incentive for hospitals, universities, and medical teaching facilities to assess and appropriately train their healthcare professionals.

The level of skill or competency of the healthcare professional can be characterized according to either continuous or ordinal scales, for example, the following three levels:
i. Competent: the minimum skill required of a practitioner to perform the procedure independently
ii. Proficient: a higher level of skill, indicating that the practitioner is capable of performing the procedure efficiently and with better outcomes
iii. Mastery: the highest level of skill, as some leaders in their specialties achieve after decades of experience.

With the platform and methods of the present invention the following can be achieved:
i. Calculation not only of a current competency score and risk score, but also the ability to forecast these scores into the future, based on upcoming clinical tasks.
ii. Decomposing medical procedures into component tasks, estimating the complexity and required skills of each task.
iii. Comparing practitioner skills to required skills, and calculating probabilities for various outcomes. These outcome probabilities can be used to make a risk score for a medical practitioner for a medical procedure.
iv. The present invention provides multiple types of performance assessment data, including both direct performance evaluations, inferred skills from training sessions, skills lab exercises, video and audio recordings of procedures or medical tasks, and data from movement-tracking wearables from medical tasks.

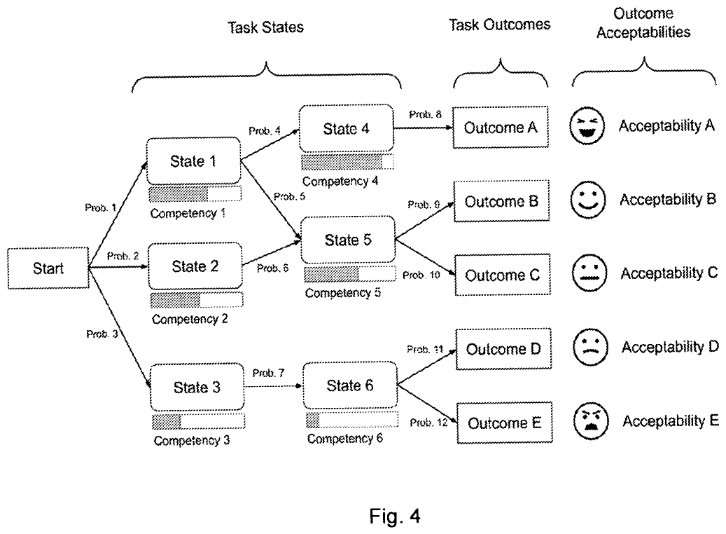

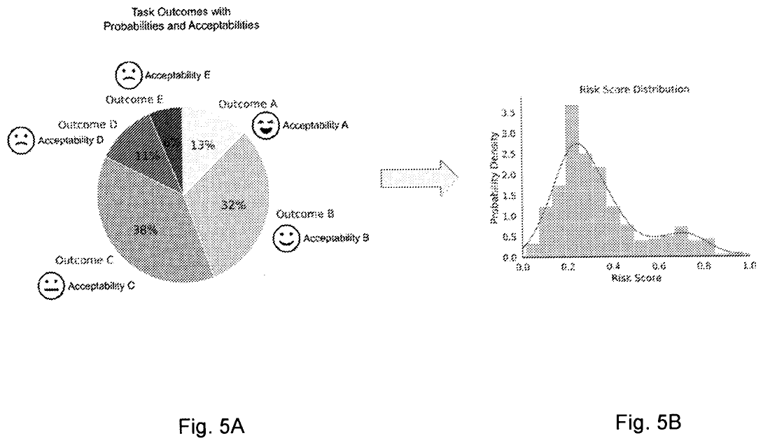

FIG. 4 is a task process model diagram and shows various exemplary paths through a procedure to completion, with the various acceptabilities associated with each possible outcome. A task, as defined previously, is a step or discrete component of a procedure. FIG. 4 illustrates how these tasks can describe the various ways of proceeding through the procedure, by the pattern of task transitions. These transition paths can create various patterns, including chains, branching patterns, and loops. For each task, the practitioner or team has a competency score, indicating their current skill level, and showing how likely they are to complete the task successfully.

Furthermore, the outcomes shown in FIG. 4 are possible outcomes of the procedure. Each outcome has a probability of occurrence, based on the task transition paths and probabilities, the practitioner's/team's task competencies and their decisions during the tasks. Each outcome also has an assigned acceptability, which is the overall assessment of satisfaction by a medical professional or organization for a task outcome. The acceptability can be based on factors, such as patient health and safety, patient peace of mind, financial cost, adherence to or deviation from standard care practice, time and resource efficiency, and patient quality-of-life level after the task is performed, etc.

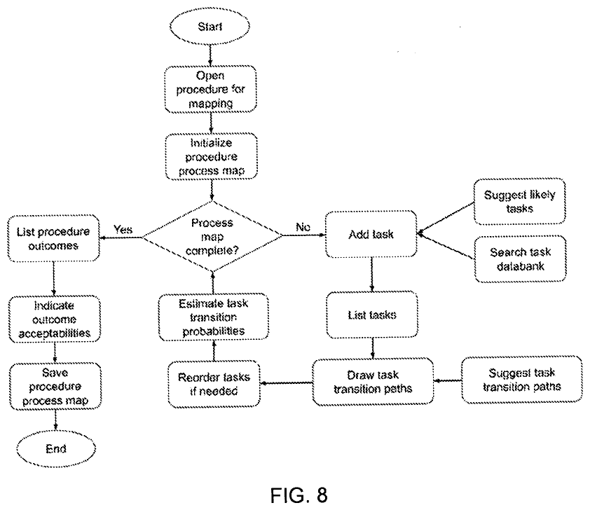

FIG. 8 shows the flow of process mapping as implemented in an embodiment of the disclosure. The process mapping flow may include the following steps:
Open procedure for mapping: The subject matter expert (SME) opens the procedure on the Firefly platform, in order to map the procedure's tasks.
Initialize procedure process map: If this is the first time that the procedure has been opened for mapping, then the Firefly system creates a new, blank procedure map to work with.
Process map complete?: The SME decides whether the procedure is described completely with its full set of tasks and outcomes, or whether he needs to add more detail.
Suggest likely tasks: The Firefly system considers the tasks added so far, and suggests appropriate tasks to add next, based on task patterns from similar procedures.
Search task databank: The SME uses the Firefly system to search for tasks. The search algorithm is smart, by ordering the most relevant tasks first, based on task patterns in similar procedures.
Add task: The SME adds a task to the procedure. This task can be either an existing task from the task suggestions or search, or a new task that the SME creates.
List tasks: Show the procedure's tasks and layout structure. One example is shown in FIG. 1, which illustrates the flow for calculating the competency score.
Suggest task transition paths: The Firefly system suggests transition paths between tasks, based on task paths in similar procedures.
Draw task transition paths: The SME can either accept the suggested paths, or draw new paths between tasks.
Reorder tasks if needed: The SME can use the graphical interface to drag tasks into new ordered positions and update the task paths appropriately.
Estimate task transition probabilities. The Firefly system predicts and suggests task transition probabilities based on paths and transition probabilities in similar procedures. The SME can either accept the suggested probabilities or manually enter the values.
List procedure outcomes: The Firefly system suggests relevant outcomes, based on outcome patterns in similar procedures. The SME can either accept the suggestions, or add new outcomes.
Indicate outcome acceptabilities: The Firefly system suggests acceptability values based on similar procedures. The SME can either accept the suggestions or enter an acceptability value. The acceptability value scale can be either a continuous range, or a level in an ordinal list.
Save process procedure map: When the SME considers that the procedure is completely mapped and described, he can click a button to save the map to the Firefly database.

The present invention is a HIPAA-compliant, web-based platform for comprehensive management of surgical research and resident education information, including operative schedules, procedural details and codes, clinical outcomes, resident and staff case assignments, performance evaluations, surgical simulation exercises, and aggregated analytics. HIPAA is an abbreviation for the Health Insurance Portability and Accountability Act of 1996, which stipulates how Personally Identifiable Information such as Protected Health Information (PHI), maintained by the healthcare and healthcare insurance industries should be protected from fraud and theft. The platform is designed to synchronize with operating room schedules and populates case logs across resident and attending case-logging databases. The platform automatically juxtaposes operating room cases with multiple types of evaluations, and matches cases with relevant educational content, for example surgical videos, journal articles, anatomical illustrations, etc. for resident preparedness. Patient-identifying data are protected and removed from analysis wherever possible.

In one aspect the present invention provides a platform wherein utilizing a computation that is based on deep learning modeling. Deep learning (also known as deep structured learning) is part of a broader family of machine learning methods. Deep-learning has been used in fields such as computer vision, speech recognition, natural language processing, machine translation, bioinformatics, drug design, medical image analysis, material inspection and board game programs. There are reports of results from some of these applications being comparable to and surpassing human expert performance.

Although the methodology of this disclosure may be based on artificial neural networks with representation learning, it is not limited to any particular algorithm, and for example, may employ any suitable machine learning model or statistical model, representative examples of which are listed below.

| Statistical models | Machine learning models | Deep learning models |
|---|---|---|
| Polynomial regression | Encoder-decoder models | Neural networks |
| Logistic regression | Attention transformer | Convolutional neural |
| Probit regression | models | networks |
| Boosting regression | Reinforcement learning | Recurrent neural networks |
| Bagging regression | Long short-term memory | Gated recurrent unit (GRU) |
| Ridge regression | (LSTM) model | networks |
| Lasso regression | Semi-supervised learning | Graph neural networks |
| Elastic net regression | | Deep reinforcement learning |
| Support vector machines | | DeepAR |
| (SVM) regression | | N-BEATS |
| Decision tree regression | | Temporal fusion transformer |
| Generalized linear models | | (TFT) |
| Autoregressive integrated | | |
| moving average (ARIMA) | | |
| Bayesian regression | | |
| (logistic, probit, etc.) | | |
| Probabilistic graphical | | |
| models | | |

At the start of each research project, custom data integrations and smart data curation tools are constructed to facilitate data aggregation and standardization of data structures. Wherever manual data entry is required, the platform uses an artificial intelligence layer to automate as much of the data-entry process as possible, with smart predictive suggestions and auto-completion of forms, trained by reinforcement learning from previous data entry patterns. Resident performance evaluations are used to fit learning curve models, to measure operative autonomy for each resident for each case type. A self-service research portal is also contemplated as part of the system, where investigators can browse posted research projects to join, or they can create their own and invite others to collaborate. The platform anonymizes and standardizes data for sharing across institutions and can be deployed multi-institutionally.

The comprehensive data platform enables near real-time monitoring and detailed analyses of operative activity and performance, and facilitates research collaboration and data collection. Potential benefits include use in tailoring curricula, and large-scale program improvement, and remediation of doctor performance.

The HIPAA-compliant web-based platform is used to track resident operative assignments and to link embedded evaluation instruments to procedure type. The platform delivered multiple evaluation types, including Ottawa O-Score autonomy evaluations. Autonomy scores are gathered across teaching faculty and combined with the residents' history of case assignments. For this analysis we focused on cholecystectomy cases. The data were entered into a logistic learning curve model, including estimates for the resident's learning lag (the number of cases needed until rapid learning), the maximum learning rate, and the autonomy limit (the maximum autonomy level we expect the resident to achieve after a large number of cases). The learning curve model included an ordinal response component, which inferred the resident's actual autonomy level from the faculty's ordinal Likert-scale ratings. It also inferred the faculty's implicit “hawk or dove” grader bias (i.e. graders who consistently graded lower or higher, respectively, than the average), while accounting for reported case complexity. The model was applied to each resident across the program, creating a learning baseline against which each individual resident can be compared to his or her peers.

We have therefore developed a learning curve model (a model based on the probability of an event occurring, based on prior knowledge of conditions related to the event) that incorporates surgical case history along with, for example, Likert-scale and Zwisch-scale evaluation data to infer and quantify resident operative autonomy. The Likert-scale is a five- or seven-point rating scale which allows an individual to express the degree to which they agree or disagree with a particular statement. The Zwisch-scale, as shown in Table 1, is a rating scale that includes anchoring behaviors and cues to advancement for residents in surgical training. The Zwisch scale is just one example of an evaluative rating scale that can be used as part of the present invention. Other rating scales can be employed, including one specifically developed for the present invention as further described below and in Table 2.

The Zwisch scale was designed to guide faculty to develop graduated autonomy for their learners during training and acts as an assessment tool, allowing faculty to understand where each trainee stands in their progression towards independence, and provides teaching strategies to help them progress. This framework is based on the “Zwisch” scale, a conceptual model that was originally used by Joseph Zwischenberger, MD, FACS, a thoracic surgeon and the chair of the department of surgery at the University of Kentucky. See, DaRosa D A, Zwischenberger J B, Meyerson S L, et al. A Theory-Based Model for Teaching and Assessing Residents in the Operating Room. JSE. 2013; 70:24-30. This model has been refined over the past several years, and now consists of four levels named “Show & Tell,” “Active Help,” “Passive Help,” and “Supervision Only.” Each level describes the amount of guidance provided by faculty to residents. The Zwisch Scale, as summarized in Table 1, describes the amount of guidance provided by faculty to residents.

TABLE 1
Summary of Zwisch Scale

| Zwisch Scale Level | Attending Behaviors | Resident Behaviors |
|---|---|---|
| Show & Tell | Performs >50% of critical portion; | Opens and closes; |
| | demonstrates key concepts, | observes and listens |
| | anatomy, and skills; explains case | during critical portion |
| | (narrates) | |
| Active Help | Leads the resident (active assist) | The above, plus actively |
| | for >50% of the critical portion; | assists (i.e. anticipates |
| | optimizes the field/exposure; | surgeon's needs); |
| | demonstrates plane/structure; | practices component |
| | coaches technical skills; coaches | technical skills |
| | next steps; identifies key anatomy | |
| Passive Help | Follows the lead of the resident | The above, plus can “set |
| | (passive assist) for >50% of the | up” and accomplish next |
| | critical portion; acts as a capable | steps; recognizes critical |
| | first assistant; coaches for polish, | transition points |
| | refinement of skills/competencies, | |
| | and safety/risk | |
| Supervision Only | Provides no unsolicited advice | The above, plus mimics |
| | for >50% of the critical portion; | independence; can work |
| | monitors progress and patient | with less experienced |
| | safety | assistant; can safely |
| | | complete case without |
| | | faculty guidance; |
| | | recovers from most |
| | | errors; recognizes when |
| | | to seek advice/help |

Furthermore, the platform of the present invention has been designed to utilize the following evaluative autonomy scale that we developed and is intended to reflect the degree of independence demonstrated to the faculty surgeon evaluator by the surgical resident. See Table 2.

TABLE 2
Evaluative Autonomy Scale

| Rating | Value | Meaning |
|---|---|---|
| I Did | 1 | I had to do. Resident did not yet have the knowledge or skill to do. |
| | | Resident requires complete hands-on guidance. |
| Steer | 2 | I had to talk them through. Resident is able to perform tasks but |
| | | requires constant direction. |
| Prompt | 3 | I had to prompt them from time to time. Resident demonstrates some |
| | | independence, but requires intermittent direction. |
| Backup | 4 | I needed to be in the room just in case. Resident has independence but |
| | | is unaware of risks and still requires supervision for safe practice. |
| Auto | 5 | I did not need to be there. Resident has complete autonomy and |
| | | independence, understands risks and performs safely, practice ready. |

The platform provides for comprehensive management of resident education information, including resident operative performance evaluations. To assess evaluation timeliness, we compared the lag time for platform-based evaluations to that of end-of-rotation evaluations. We also assessed evaluation compliance, based on a time threshold of 5 days for platform evaluations and 2 weeks for end-of-rotation evaluations.

Evaluation of performance is an essential responsibility of the teaching faculty members of any surgical residency. Although the Accreditation Council for Graduate Medical Education (ACGME) explicitly defines this responsibility in section V of the Common Program Requirements, specific evaluation instrument types, specific methods to achieve timely completion, control of evaluation quality, and effective use as tools to facilitate positive development are areas where training programs have enormous latitude to utilize innovative methods. The use of evaluation as a feedback tool is vitally important in surgical training, and although published evidence of obstacles to achievement of effective feedback are scant, this issue is nonetheless frequently cited in the context of time pressures and conflicting responsibilities experienced by faculty members. There is agreement that absence of effective feedback is an impediment to high quality medical training, and that frequent evaluations are required for effective resident assessment. See, Anderson P A. Giving feedback on clinical skills: are we starving our young? J Grad Med Educ. 2012; 4:154-158.; Williams R G, Verhulst S, Colliver J A, Sanfey H, Chen X, Dunnington G L. A template for reliable assessment of resident operative performance: assessment intervals, numbers of cases and raters. Surgery. 2012; 152: 517-524. https://doi.org/10.1016/j.surg.2012.07.004. discussion 524-7 Epub 2012 Aug. 28; Dougherty P, Kasten S J, Reynolds R K, Prince M E, Lypson M L. Intraoperative assessment of residents. J Grad Med Educ. 2013; 5:333-334. https://doi.org/10.4300/JGME-D-13-00074.1; Williams R G, Swanson D B, Fryer J P, et al. How many observations are needed to assess a surgical trainee's state of operative competency? Ann Surg. 2019; 269:377-382. https://doi.org/10.1097/SLA.0000000000002554.; and (5) Fryer J P, Teitelbaum E N, George B C, et al. Effect of ongoing assessment of resident operative autonomy on the operating room environment. J Surg Educ. 2018; 75:333-343. https://doi.org/10.1016/j.jsurg.2016.11.018. Epub 2017 Mar. 28.

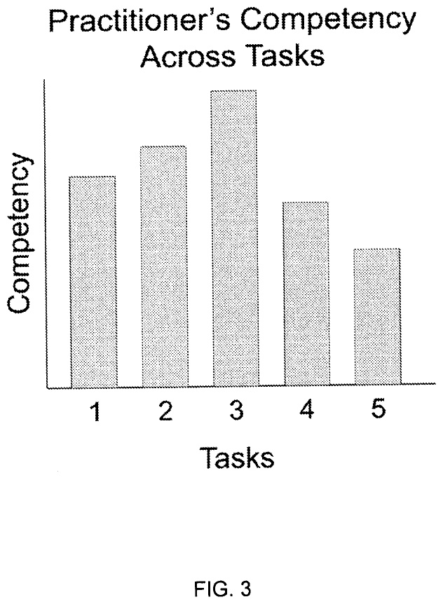

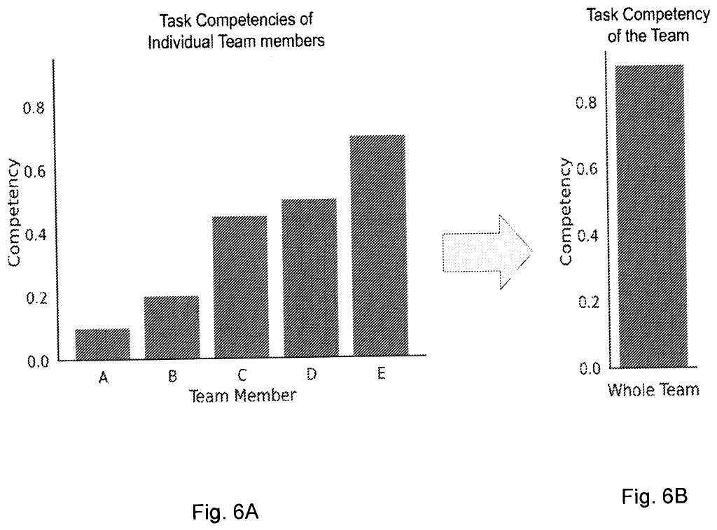

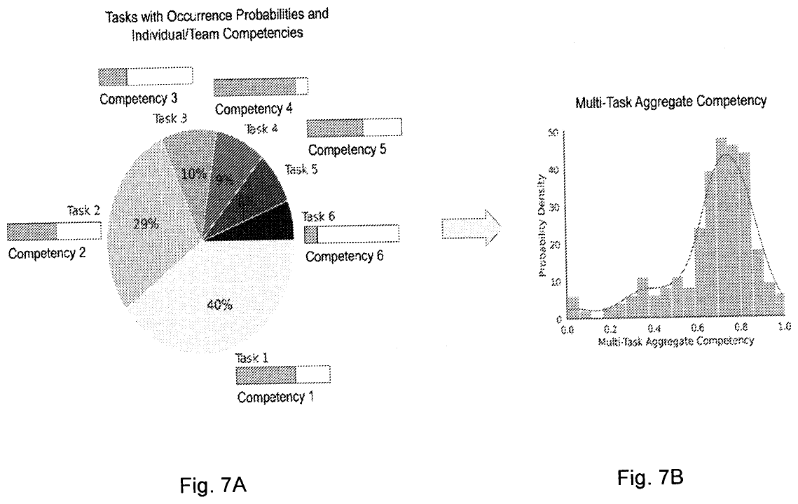

As shown in FIG. 3, a competency score for each task of an overall procedure is computed and assigned to each individual practitioner. Each medical practitioner and team will naturally encounter a wide variety of tasks in the course of their professional role, and will be expected to become competent to handle these various tasks. By combining each task with its frequency of occurrence and the with the practitioner's or team's competence in that task, a multi-task aggregate competency may be calculated. FIGS. 6A and 6B show an example of how individual or team competencies for particular tasks may be integrated into a multi-task aggregate competency distribution. A probability distribution such as shown in FIG. 6B may be used as a snapshot of a practitioner's overall competency in handling the tasks typically presented in her/his job or how skilled a team is at their collective job or responsibility. In other embodiments, this multi-task aggregate competency may be indicated as a single value, rather than as a probability distribution.

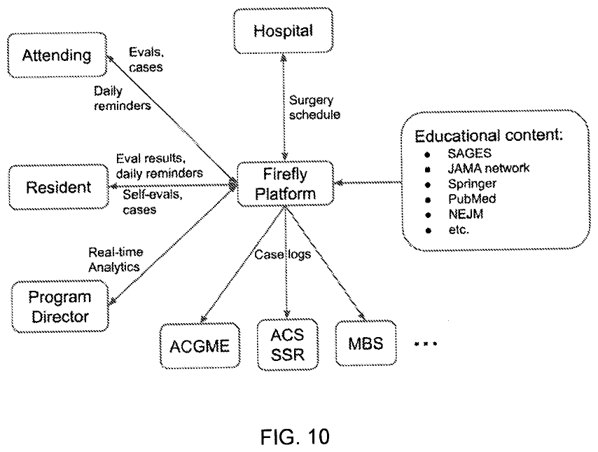

Additionally, the platform connects multiple systems and uses, as is exemplified in FIG. 10. FIG. 10 shows the platform's data integration and user role architecture. Some of the important components can be summarized as follows:
1. The Firefly™ platform connects with the hospital's data system to access relevant clinical data and schedules, for example the operative schedule for a surgery team.
2. The platform connects with, indexes, and profiles large amounts of educational content, for example journal articles, anatomy diagrams, and medical procedure videos. The Firefly™ targeted education system associates each piece of content with relevant medical activities, using techniques including machine learning and natural language processing.
3. The platform connects with case logging systems for automated storage and reconciling of a provider's clinical experience. The Firefly™ case reconciling system performs data curation and automatically identifies and merges duplicate case records.
4. The platform searches and assembles relevant information for various types of users, including a comprehensive real-time dashboard of clinical and educational information for a residency program director, medical tasks and evaluations for residents, and medical tasks and evaluations for attendings.
5. Other features of the method and platform include: evaluations and learning profiles, targeted education, case logging, and case analytics.

Individual residents (practitioners) and programs must satisfy and complete certain mandated requirements. See Nygaard, Rachel M., Samuel R. Daly, and Joan M. Van Camp. “General surgery resident case logs: do they accurately reflect resident experience?.” Journal of surgical education 72.6 (2015): e178-e183, which notes a 24.2% discrepancy between cases log into ACGME and cases residents participated in based on electronic medical records (EMR). The most common reason for this discrepancy is that 9.6% “forgot to log”, which highlights inconsistent logging practices amongst residents. On the other hand, it has been shown that semi-automation of procedure logging in emergency medicine leads to 168% increase in procedure logging. See, Seufert, Thomas S., et al. “An automated procedure logging system improves resident documentation compliance.” Academic Emergency Medicine 18 (2011): S54-S58.

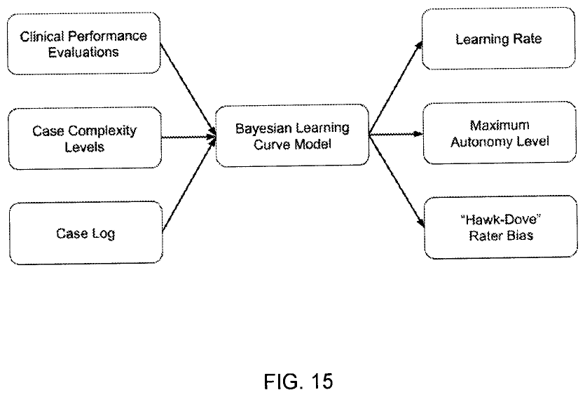

FIG. 15 illustrates the flow and logic of the system and methods of the present invention. The data flow and steps can be summarized as follows:
1. Gather the clinical performance evaluations for the practitioner to be modeled, as well as for her/his peers. The evaluations can be of different types. The model can use partial information to infer missing data for each step of clinical procedures.
2. Gather and standardize everyone's case logs (records of a practitioner's clinical encounters and procedures).
3. Based on the evaluation and clinical data, estimate the clinical complexity level for each case. The case complexity is important for knowing how informative each case is for estimating the practitioner's expertise level, i.e., the competency score. If the case is much too easy or too hard for the practitioner, then it will not contribute much to our understanding of the practitioner's expertise level.
4. Estimate the prior distributions for relevant model parameters: hawk-dove rater bias (whether the teacher typically gives low or high grades) for each person who completed an evaluation (typically a teaching faculty member), and the practitioner's learning rate (how fast the practitioner learns with each procedure) and maximum autonomy/expertise level (how independent we predict the practitioner will be after a large number of procedures). Do this for each component task of each procedure.
5. Run a Markov Chain Monte Carlo (MCMC) statistical sampling method to estimate the posterior distributions of the learning curve parameters.
6. From the posterior samples, infer the practitioner's learning curve for each step of each procedure. The model will generate a distribution for each parameter: the learning rate, max autonomy level, and the hawk-rater biases of each of her/his teachers. And finally,
7. Compare each practitioner to her/his peers, in order to calculate her/his rank and percentile during the learning process. The results of this comparison will show whether the practitioner is ahead or behind her/his peers in the learning process.

The performance advantages and features of the present invention include:

Automated data entry and efficient workflow in a clinical setting.

Advanced statistical modeling system to quantify a medical provider's competence or expertise with a medical procedure.

A system to index, match, and suggest educational content for the medical practitioner based on her/his clinical/surgical schedule, specialty, and current level of competency. And also a system to characterize the clinical/surgical experience and performance of a group of medical professionals, and to normalize the expertise (competency) level of each professional according to that of his/her matched peers.

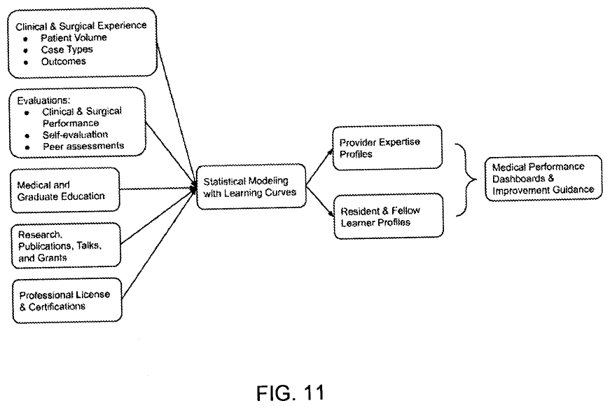

FIG. 11 shows the data flow and processing system for quantifying medical expertise (competency) and constructing medical learner profiles. The data flow and steps can be summarized as follows:
1. For each medical practitioner, gather clinical and surgical experience, including patient volume, case types with procedure information, and patient outcomes.
2. Gather evaluation data, including evaluations of clinical and surgical performance, self-evaluations, and peer assessments.
3. In addition to clinical information, also gather available data on medical and graduate education, research outcomes (e.g. publications, posters, conference talks, and grants), and professional licenses and certifications.
4. Perform the statistical modeling and construction of learning curves on each relevant medical task and procedure, as described above.
5. From these learning curves, construct medical expertise profiles and learner profiles, to summarize each practitioner's expertise levels (competency scores) and to compare to relevant peer groups.
6. Assemble the expertise (competency) and learning profiles into a live dashboard for tracking clinical activities and learning rates. From the Firefly™ (the present invention) targeted education system, include targeted educational content, learning milestones, and suggestions, as appropriate for each medical learner.

In one exemplary application, the platform combined disparate data across 37 institutions, comprising 47 surgical departments and 100 surgical services, aggregating 278,410 surgical operative cases with 340,128 associated procedures, and 493,807 case assignments. From these, 184,318 resident cases were logged with the ACGME, and 17,969 cases were logged to the American College of Surgeon's (ACS) Surgeon Specific Registry. The platform helped the teaching faculty submit 4,285 resident operative performance evaluations, enabling the construction of 165 procedure-specific learner profiles. Additionally, the platform aggregated 54, 126 data points from resident surgical simulation exercises, including virtual reality laparoscopic simulations.

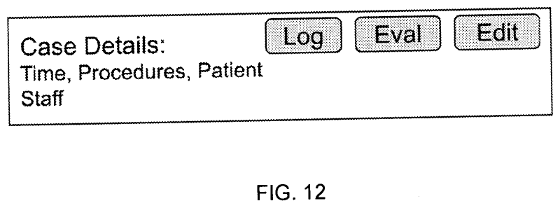

Also, contemplated with the present invention are the computer systems, associated hardware, servers, software, code, and algorithms necessary for compiling, preprocessing, curating, storing, analyzing, and manipulating the inputted information, as well as conducting the various searches, projections, simulations, and outputs. As illustrated in FIG. 12, which shows an example of a user interface for the methods and systems of the present invention, the case logging and evaluations may be integrated into the schedule via a user interface. The user interface can be a graphical user interface (GUI). As is well known, a GUI is a type of user interface that allows users to interact with electronic devices. The interface can provide for graphical icons and audio indicators such as a primary notation, instead of text-based user interfaces, typed command labels or text navigation. In other embodiments of the present invention, the interface can be a command-line interface or a menu driven interface. There is quick case logging with smart Common Procedural Technology (CPT) suggestions. CPT is a formal way of assigning codes to medical procedures, and is commonly used for billing, as insurance companies have predetermined amounts they reimburse for each code. These codes are useful for case logging, for the doctor to be more precise about the procedures performed.

The systems and methods allow for bidirectional syncing with ACGME and ACS case logs and automatically fills in case details from the schedule, using machine learning to search and suggest CPT codes. The system also has the capability to learn from case logging patterns across a department. Advantages include: the rapid smart adding of cases such that the surgeons log their cases very quickly (10 seconds) and without delay (the same day). We have demonstrated that residents log their cases earlier (more than 5 days earlier) than into ACGME. Also, there is an advantage of early logging behavior for the platform versus ACGME database. We also have found that there is a preference amongst surgeons to use the system of the present invention versus ACGME.

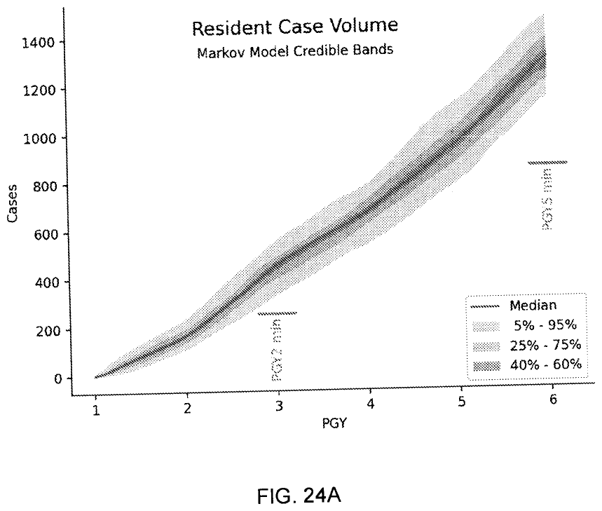

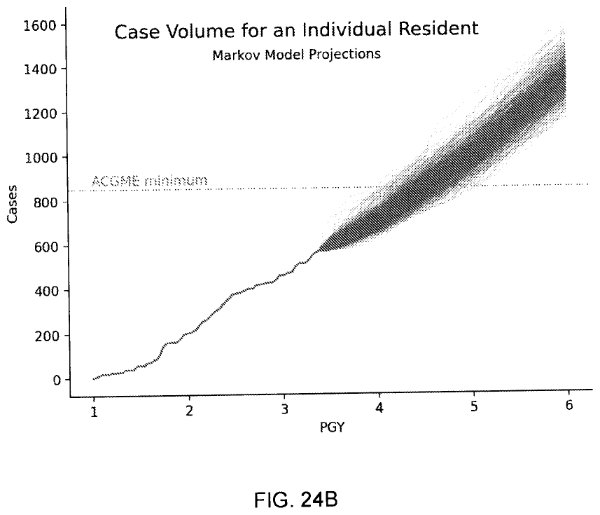

The systems and methods of the present invention provide for a live analytics dashboard which can be synchronized with ACGME reports. This feature allows residents to explore and compare case mix. There is also the capability to compare case experience across residents. The benefit of these features is the ability to predict resident case volume. See FIGS. 24A and 24B which illustrate how the methods and systems of the present invention are useful for predicting case volume. FIG. 24A shows total resident (practitioner) case volume. FIG. 24B shows the case volume for an individual resident (practitioner).

Yet another feature is the ability to have multiple evaluations delivered on a desktop and phone.

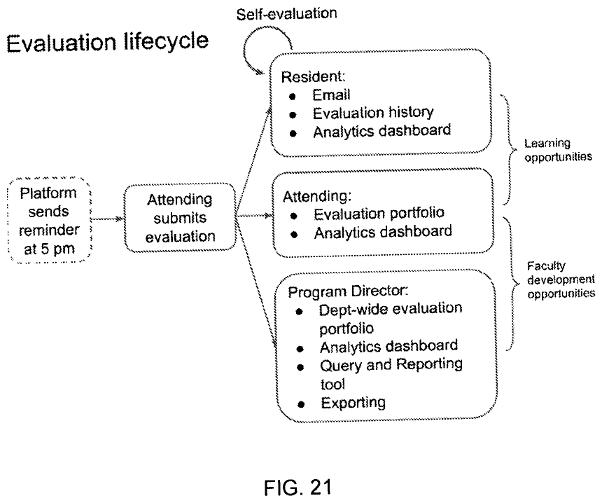

FIG. 21 shows the evaluation life cycle diagram for the methods and systems of the present invention. Either at the end of each teaching case or at the end of the workday (depending on the user's notification preferences), the platform sends an evaluation request to the teaching attending. Once the attending then completes and submits the evaluation, the platform sends the evaluation to provide the resident (practitioner) with immediate performance feedback. The evaluation is also inserted into the attending's personal evaluation portfolio and dashboard, as well as the program director's department-wide evaluation portfolio and analytics dashboard. This dashboard provides a live view of all evaluation activity across the department, along with data query and exploration tools for visualizing and analyzing the data.

The methods and systems of the present invention provide advantages for resident evaluation. The attending surgeons evaluate quickly and without delay. This enables residents to get feedback early, when it is most helpful and relevant throughout their rotations. Facilitated by the platform, attendings typically complete their evaluations within one minute. Because the process is quick, attendings submit their evaluations within a few days of the case, rather than postponing the task. Also, the attending surgeons evaluate quickly for multiple evaluation types. We have demonstrated that platform evaluations arrived 35 days earlier. Because the platform provides convenient prompts and reminders for the evaluations, as well as optimized workflow to make the evaluation process quick, the attendings complete their evaluations over a month earlier on the platform than they had traditionally done without it. On Firefly™, approximately 95% of the evaluations were submitted within a few days of each teaching case.

The analytics dashboard can show live evaluation statistics, resident learning trends, and even has the ability to show a system-level view of evaluations across a department.

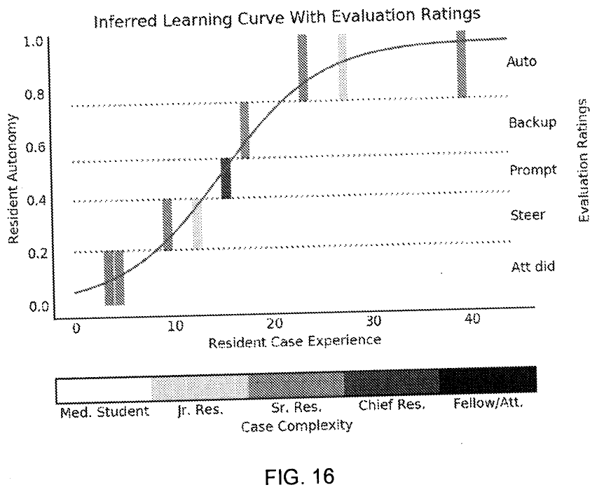

Other features of the systems and methods of the present invention include the capability for modeling for resident learning and autonomy. See FIG. 16. This shows the resident autonomy level using the evaluation scale of Table 2 on the y-axis versus case complexity on the x-axis. The title of the doctor (practitioner) is also shown, i.e., medical student, junior resident, senior resident, chief resident, and fellow/attending surgeon. There is the capability for self-assembling consensus evaluations, links to educational content from the schedules, a targeted education library of curated content, and an active research feature with a content recommendation engine.

Data Security and HIPAA Compliance and Protected Health Information (PHI). The systems and methods of the present invention having the important advantage of being HIPAA compliant. The invention utilizes strong encryption for all data connections and the databases and can be securely hosted on the could. The invention allows for two-factor authentication with routine penetration testing and risk assessments on data system infrastructure. A department administrator can be assigned to manage users and data access. Also, any PHI can be optional on the platform.

The system can be securely protected from the provider and utilizes blind storage for encrypted PHI where it cannot decrypt or read surgeon-encrypted data, because the PHI is encrypted locally on the surgeon's computer with secret encryption keys. To decrypt data, potential hackers would have to break into the system provider and hospital data systems simultaneously.

Also, the system has a very tight IT footprint. By default, the system operates independently without any IT support or data integration burden from the hospital. The system optionally can accept a secure data feed of the surgical schedule, which saves the surgeons from having to type in their case information for case logging.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1

### Educational Information Management Platform Improves the Surgical Resident Evaluation Process.

### Objective:

We sought to increase compliance and timeliness of surgery resident operative evaluation, by providing faculty and residents with a platform linking evaluation to analytics and machine-learning-facilitated case logging. See, Thanawala, R., Jesneck, J. and Seymour, N. E., 2018. Novel Educational Information Management Platform Improves the Surgical Skill Evaluation Process of Surgical Residents. Journal of Surgical Education, 75(6), pp.e204-e211.

### Design:

We built a HIPAA-compliant web-based platform for comprehensive management of resident education information, including resident operative performance evaluations. To assess evaluation timeliness, we compared the lag time for platform-based evaluations to that of end-of-rotation evaluations. We also assessed evaluation compliance, based on a time threshold of 5 days for platform evaluations and 2 weeks for end-of-rotation evaluations.

### Participants:

23 attendings and 43 residents for the platform cohort. 15 services and 45 residents for the end-of-rotation cohort.

The desired outcome of surgical education is the achievement of defined competencies including the ability to function with a high degree of autonomy in the operating room. It is critically important to evaluate operative performance in effective ways in order to make well informed statements on this (1). Evaluations are most effective when they are completed and made available to the learner without delay (2,3). However, completing individual evaluations in the desired time frame requires frequent data entry and places a time and work burden on surgical educators (4,5). Large clinical productivity expectations, burdensome non-clinical workloads, and the risk of burnout that accompanies ever-increasing demands for time that is in short supply are threats to the quality of educational activities such as resident performance evaluations. Other forms of practice data entry can also be affected (6,7), including keeping up with required clerical tasks and records of operative cases (8,9).

Several strategies have been used to ease the process of operative evaluations, including mobile applications (10), web-based applications (11-13), and residency information management systems. These innovations might improve the process of evaluation submission in their specific niches, but demonstration of this is challenging. We sought to address the evaluation submission process with an additional strategy that centralizes data entry in a comprehensive platform, where an evaluation is accessed along with other tasks that utilize some of the same data stream. Combining related tasks into one workflow increases ease of use and avoids the cognitive burden of navigating to isolated systems in a more complicated workflow (6). Such a comprehensive system can take advantage of experience in other established data-intensive fields, such as engineering and computer science, to optimize workflow, improve usability, decrease cognitive burden of frequent tasks, and create positive feedback loops for beneficial user habits (8). It was our aim to add value to the process of surgical skills evaluation by providing faculty and resident participants in the evaluation process a platform linking evaluation to case logging, and thereby improving compliance, timeliness, and sustainability of evaluation practice.

### Materials and Methods

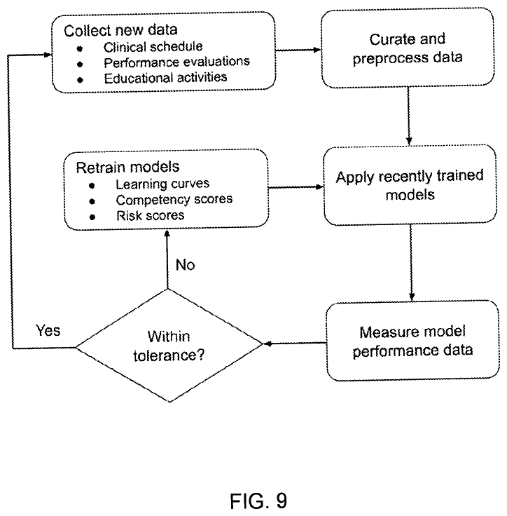

We built a HIPAA-compliant web-based platform for comprehensive management of resident education information including performance evaluations. To optimize evaluation workflow, the platform synced with the institution's operating room (OR) schedules and automatically merged patient and case data, including coding description of operative procedures, attending surgeon, resident surgeon, date of operation, and OR location. These combined data were delivered in real-time in an editable system that included case schedule pertinent to the user, specific resident case assignments, case logging functionality for residents and attendings, and finally, resident operative performance evaluations (FIG. 9). Case logging workflow benefited from using the scheduled case information to limit manual data entry. Case information was validated (or edited) following a case when the platform was accessed. Logging data were then automatically inserted into the Accreditation Council for Graduate Medical Education (ACGME) case log for residents and the American College of Surgeons-Surgeon Specific Registry (ACS-SSR) for attendings. Additionally, the platform learned from previous case logging patterns to provide smart search and automated suggestions for Current Procedural Terminology (CPT) codes (14) using machine learning. For each operation with a resident, the platform offered to the attending a resident operative performance evaluation with a single mouse click or screen tap. Evaluations consisted of a slightly modified Ottawa O-Score instrument rating of operative autonomy on a five-point Likert scale for 12 items along with the option to insert narrative comments (15). Evaluation results were displayed in a real-time analytics dashboard for the evaluating attendings, evaluated residents, and program director. For ease of use, the platform was mobile friendly, so that attendings could complete evaluations from their smartphones. The platform automatically sent attendings daily reminder emails to complete evaluations, and upon completion it immediately pushed evaluation results to the residents. The real-time evaluation status was embedded into the surgical schedule beside each case, facilitating rapid progress through multiple evaluations, and reminding evaluators to complete all evaluations.

Timeliness of evaluation submission was used as the principal measure of the platform's usability. Understanding that broader evaluations of resident performance on individual rotations was a different construct, we did compare timeliness of platform-based evaluations with end-of-rotation evaluations delivered to evaluators via the Program's overall information management package (New Innovations, Uniontown, OH) (16). For the platform, we measured timeliness by the lag in number of days between the operation and the evaluation submission. For end-of-rotation evaluations, timeliness was the lag in number of days between the end of rotation and the evaluation submission. We compared median lag times using Mood's median test (17), and compared mean lag times using unpaired t-test with unequal variance (18). Using these lag values, we applied thresholds to define evaluation compliance. We defined compliance for the platform evaluations as within five days of the case and for the end-of-rotation evaluations as within two weeks of the end of rotation. We compared compliance rates and tested for statistical significance by using bootstrap sampling. We also recorded the hour of day when attendings submitted their evaluations, in order to understand how the evaluation process fits into their daily workflow.

### Results

358 platform evaluations were completed by 23 attendings for 43 residents for March through October 2017. 610 end-of-rotation evaluations by 15 attendings for 45 residents were used for comparison (September 2015 through June 2017). 41.3% of platform evaluations were completed within 24 hours of the operation (16.5% in 6 h, 33.3% in 12 h, 62.2% in 48 h).

In the first six weeks (March 1 through April 12) 4.5±3.7 evaluations were completed per week compared to 18.8±5.8 in the last six weeks (September 18 through October 31). Evaluation lag times improved with use of the platform, both for median lag of 35 days earlier (1±1.5 days platform, 36±28.2 days traditional, p<0.0001) and a mean lag of 41 days earlier (3.0±4.7 days platform, 44.0±32.6 days traditional, p<0.0001).

We defined the timeliness of evaluations to be the percent of evaluations submitted by a given lag time. The attendings submitted almost all of the evaluations within 5 days for the platform evaluations, and within 140 days for the end-of-rotation evaluations.

From the timeliness, we used time thresholds to define evaluation compliance. The compliance was significantly higher for the platform evaluations (79%±2%) than for the end-of-rotation evaluations (16%±1%) (p-value<0.00001). The attendings filled out the platform evaluations quickly, with 49% within one minute and 75% within two minutes. Attendings typically submitted evaluations throughout the day, 81% during main operating hours 07:00 h to 18:00 h and 19% during evening hours. 24% of evaluations were completed within 3 hours after automated daily email reminders were sent at 17:00 h.

### CONCLUSIONS

Our comprehensive platform facilitated faculty compliance with evaluation requirements and timeliness of availability of performance information (often in real-time or near real-time) for both residents and residency leadership. The platform aimed to improve the process of evaluation and the evaluator experience by three strategies: 1) limiting manual data entry by pre-populating relevant data, 2) focusing on ease-of-use to streamline workflow, and 3) increasing value for evaluators by combining evaluation with case logging connected to achievement of Maintenance of Certification (MOC) requirements. Platform features related to the latter strategy eliminated the need to enter case details or to select the assigned resident, and made any editing of these details simple. The platform's ease-of-use made initial instruction simple with the primary focus on login procedure and familiarity with the evaluation instrument. Based on our results, most importantly the rapid completion of the evaluations, the goal of facilitating the resident operative evaluation process was met. This process rapidity increased the likelihood that feedback was either delivered face-to-face, or reached the resident soon enough to be meaningful. This effect was not measured but is nonetheless one of the major goals: To enable positive feedback loops in user interaction in order to promote compliance and engagement. Although we did not survey user subjective reactions, we propose that the platform actually reduced the work burden that would have been experienced if the evaluation and logging tasks had been performed outside the common platform, and that this likely facilitated the prompt completion of evaluations we observed. The provision of automatic populating of data fields with case information was a major factor in reducing keyboard, mouse, and screen interactions to a minimum.

Longitudinally performed evaluations can serve as a means to demonstrate resident learning and improvement during a specific rotation, or over longer periods of time. Although rotation evaluations are a substantially different construct in that these require consensus input from multiple faculty members and are not deemed “complete” until all input is received, they represent a task at the opposite end of the effort spectrum. Not surprisingly, we observed rates of completion of rotation evaluation that reflected gradual accumulation of information which, while useful, delayed availability in many cases to a degree that almost certainly diminished their usefulness as feedback tools. There is an opportunity to look at this process differently based on the benefits of optimizing workflow. The platform can be used to automate the merging of evaluation information across faculty members, in order to create self-assembling consensus evaluations. The platform can gather case-based evaluations for a resident, and then present a summary of these evaluations to the evaluating faculty member as a reminder of the resident's performance over the rotation period. The faculty member can submit a streamlined end-of-rotation evaluation, including overall performance scores, feedback, and suggestions. As each evaluating faculty member completes the evaluations, the platform can assemble them into an inferred consensus evaluation, showing the distribution of the resident's performance scores as well as aggregated comments from the teaching faculty.

A positive feedback loop is further enabled by the analytics dashboard. The platform's real-time analytics dashboard presents tangible evaluation results so that they are easy to find and are understandable. Residents can see evaluative scores improve with practice and experience, incenting further practice and experience. Faculty members can see how many evaluations they have completed compared to their peers, incenting greater compliance with program evaluation needs. The dashboard provides the program director with an aggregated view of the evaluation results, in order to monitor resident progress and identify situations where directed action might be required to help with skills development.

Other commonly-used surgical resident evaluation tools exist, such as System for Improving and Measuring Procedural Learning (SIMPL) (19), and have similar goals of making evaluation process more convenient and accessible. One of the chief differences between these approaches and our platform is that our platform is agnostic to evaluation type. As we move forward with platform-based evaluation applications, it will be possible to capitalize on this to integrate evaluation instruments with a variety of intended purposes, including comparison studies or use of “best-practice” tools as they are developed. We have integrated Operative Performance Rating System (OPRS) evaluations required by the American Board of Surgery as well as resident self-efficacy evaluations (20), which are being compared to attendings' assessments. Another difference is that, instead of relying on residents to request being evaluated, the platform integrates with the hospital schedule and case assignments, and therefore it automatically detects when an evaluation event should occur. The schedule integration enables the platform to protect attendings and residents from forgetting to evaluate by targeted, case-specific daily reminder emails. The automated end-of-day and subsequent daily reminder emails might be a help to time-taxed attendings who wish to complete evaluations and log their cases, but we also found that these were not necessary for the majority of cases.

With the integrated schedule, the platform shows evaluation status as buttons embedded into the surgical schedule. This convenience saves the evaluator from having to track evaluations manually, in order to know whether any residents still require evaluations for the week's cases.

The schedule integration also enables convenient look-up of case details, which can help to jog the memory of the residents' actions and facilitate rapid completion of the performance scoring. Unlike with stand-alone evaluation tools, the evaluator does not need to describe the case procedure details or select the intended resident, since these data are pulled from the schedule.

Perhaps the platform's most unique differentiator is its integration with case logging systems. Since the ACGME makes case logging mandatory for residents, and the American Board of Surgery requires an accounting of cases for MOC, the platform's ability sync with the residents' ACGME case log and the ACS-SSR makes reduced interface burdens a benefit that can be experienced on a nearly daily basis. We have now learned that, when the process is sufficiently convenient, busy attending surgeons will integrate it into their daily post-operative workflow. Due to the streamlined evaluation process and extra incentive of case logging, throughout the study we saw increasing participation, whereas only 35% of attendings and 36% of residents reported using SIMPL after 20% of cases (21). We are also expanding the platform to other surgical subspecialties by integrating with their case logging systems, including the Mastery of Breast Surgery case log (22). By synthesizing the information within the platform, our long-term goal is to measure the impact of the evaluations on resident operative performance and to measure learning rates for individual residents and individual operations.

Referring to the figures, FIG. 9 shows how the platform system architecture connects to disparate data systems, such as the hospital schedule and case logging systems, and surgery residents and teaching faculty. Although not shown, the platform's Case History page would show a surgeon's queue of recent cases, along with relevant actions for case logging and evaluating. The Case History page can include colored action buttons to indicate those actions that still needs to be performed. A “queued” log state means that the case has been logged into Firefly™ and is queued for automatic logging into an external case log, such as the ACS SSR or Mastery of Breast Surgery (MBS).

### REFERENCES FOR EXAMPLE 1 ABOVE

1. Williams, R. G, Kim, M. J., and Dunnington, G. L, 2016. Practice guidelines for operative performance assessments. Annals of surgery, 264(6), pp. 934-948.
2. Karim, A. S., Sternbach, J. M., Bender, E. M., Zwischenberger, J. B. and Meyerson, S. L., 2017. Quality of Operative Performance Feedback Given to Thoracic Surgery Residents Using an App-Based System. Journal of surgical education, 74(6), pp.e81-e87.
3. Roberts, N. K., Williams, R. G., Kim, M. J. and Dunnington, G. L., 2009. The briefing, intraoperative teaching, debriefing model for teaching in the operating room. Journal of the American College of Surgeons, 208(2), pp. 299-303.
4. Dougherty, P., Kasten, S. J., Reynolds, R. K., Prince, M. E. and Lypson, M. L., 2013. Intraoperative assessment of residents. Journal of Graduate Medical Education, 5(2), pp. 333-334.
5. Roberts, N. K, Brenner, M. J, Williams, R. G, Kim, M. J. and Dunnington, G. L., 2012. Capturing the teachable moment: a grounded theory study of verbal teaching interactions in the operating room. Surgery, 151(5), pp. 643-650.
6. Raj M Ratwani, Rollin J Fairbanks, A Zachary Hettinger, Natalie C Benda; Electronic health record usability: analysis of the user-centered design processes of eleven electronic health record vendors, Journal of the American Medical Informatics Association, Volume 22, Issue 6, 1 Nov. 2015, Pages 1179-1182, https://doi.org/10.1093/jamia/ocv050.
7. Sittig, D. F. and Singh, H., 2011. Defining health information technology-related errors: New developments since To Err Is Human. Archives of internal medicine, 171(14), pp. 1281-1284.
8. Johnson C M, Nahm M, Shaw R J, et al. Can Prospective Usability Evaluation Predict Data Errors? AMIA Annual Symposium Proceedings. 2010; 2010:346-350.
9. Shanafelt, T. D., Dyrbye, L. N., Sinsky, C., Hasan, O., Satele, D., Sloan, J. and West, C. P., 2016, July. Relationship between clerical burden and characteristics of the electronic environment with physician burnout and professional satisfaction. In Mayo Clinic Proceedings (Vol. 91, No. 7, pp. 836-848). Elsevier.
10. Bohnen, J. D., George, B. C., Williams, R. G., Schuller, M. C., DaRosa, D. A., Torbeck, L., Mullen, J. T., Meyerson, S. L., Auyang, E. D., Chipman, J. G. and Choi, J. N., 2016. The feasibility of real-time intraoperative performance assessment with SIMPL (system for improving and measuring procedural learning): early experience from a multi-institutional trial. Journal of surgical education, 73(6), pp.e118-e130.
11. Wagner, J. P., Chen, D. C., Donahue, T. R., Quach, C., Hines, O. J., Hiatt, J. R. and Tillou, A., 2014. Assessment of resident operative performance using a real-time mobile Web system: preparing for the milestone age. Journal of surgical education, 71(6), pp.e41-e46.
12. Sehli, D. N., Esene, I. N. and Baeesa, S. S., 2016. A proposed Resident's operative case tracking and evaluation system. World neurosurgery, 87, pp. 548-556.
13. Hartranft, T. H., Yandle, K., Graham, T., Holden, C. and Chambers, L. W., 2017. Evaluating Surgical Residents Quickly and Easily Against the Milestones Using Electronic Formative Feedback. Journal of surgical education, 74(2), pp. 237-242.
14. American Medical Association: CPT-Current Procedural Terminology. www.ama-assn.org/ama/pub/physician-resources/solutions-managing-your-practice/coding-billing-insurance/cpt.pagewww.ama-assn.org/ama/pub/physician-resources/solutions-managing-your-practice/coding-billing-insurance/cpt.page
15. Gofton, W. T., Dudek, N. L., Wood, T. J., Balaa, F. and Hamstra, S. J., 2012. The Ottawa surgical competency operating room evaluation (O-SCORE): a tool to assess surgical competence. Academic Medicine, 87(10), pp. 1401-1407.
16. New Innovations. https://www.new-innov.com/. Accessed Apr. 1, 2018.
17. Corder, G. W. & Foreman, D. I. (2014). Nonparametric Statistics: A Step-by-Step Approach, Wiley. ISBN 978-1118840313.
18. Coombs, W. T., Algina, J. and Oltman, D. O., 1996. Univariate and multivariate omnibus hypothesis tests selected to control Type I error rates when population variances are not necessarily equal. Review of Educational Research, 66(2), pp. 137-179.
19. George, B. C., Teitelbaum, E. N., Meyerson, S. L., Schuller, M. C., DaRosa, D. A., Petrusa, E. R., Petito, L. C. and Fryer, J. P., 2014. Reliability, validity, and feasibility of the Zwisch scale for the assessment of intraoperative performance. Journal of surgical education, 71(6), pp.e90-e96.
20. de Blacam, C., O'Keeffe, D. A., Nugent, E., Doherty, E. and Traynor, O., 2012. Are residents accurate in their assessments of their own surgical skills?. The American Journal of Surgery, 204(5), pp. 724-731.
21. Eaton, M., Scully, R., Yang, A., Schuller, M., Smink, D., Williams, R., Bohnen, J., George, B., Meyerson, S., Karmur, A., Fryer, J., 2018, Value and barriers to use of a SIMPL tool for resident feedback, paper presented to Surgical Education Week, Association of Program Directors in Surgery, Austin, TX, May 2018
22. Mastery of Breast Surgery, the American Society of Breast Surgeons https://masterybreastsurgeons.org

### Example 2

### Education Management Platform Enables Delivery and Comparison of Multiple Evaluation Types

The following are summary points from this Example 2:

The education management platform demonstrated a convenient method to deliver multiple operative evaluations intelligently matched to the appropriate operations. The platform delivered multiple appropriate evaluations together for the same cases provides an opportunity to study resident performance across operative evaluations. The platform-based evaluations can be completed in under a minute with an additional 1-2 minutes if comments are added.

The purpose of the work described in this example is for making multiple surgical evaluation instruments available when needed for appropriate clinical situations, including specific case types, presents some challenges that might impede convenient usage. We evaluated the impact of simultaneously delivering two evaluation instruments via a secure web-based education platform to test how easily these could be completed by faculty surgeon evaluators when rating resident operative performance, and how effectively the results of evaluation could be analyzed and compared, taking advantage of a highly integrated management of the evaluative information.

### Methods:

We built a HIPAA-compliant web-based platform to track resident operative assignments and to link embedded evaluation instruments to procedure type. The platform matched appropriate evaluations to surgeons' scheduled procedures, and delivered multiple evaluations, including Ottawa O-Score autonomy evaluations and Operative Performance Rating System (OPRS) evaluations. Prompts to complete evaluations were made through a system of automatic electronic notifications. We compared the time spent in the platform to achieve evaluation completion. For those cases for which faculty completed both O-Score and OPRS evaluations, correlation was analyzed by Spearman rank test. Evaluation data were compared between PGY levels level using repeated measures ANOVA.

Evaluation of performance is an essential responsibility of the teaching faculty members of any surgical residency. Although the Accreditation Council for Graduate Medical Education (ACGME) explicitly defines this responsibility in section V of the Common Program Requirements, specific evaluation instrument types, specific methods to achieve timely completion, control of evaluation quality, and effective use as tools to facilitate positive development are areas where training programs have enormous latitude to utilize innovative methods. The use of evaluation as a feedback tool is vitally important in surgical training, and although published evidence of obstacles to achievement of effective feedback are scant, this issue is nonetheless frequently cited in the context of time pressures and conflicting responsibilities experienced by faculty members. There is agreement that absence of effective feedback is an impediment to high quality medical training (1), and that frequent evaluations are required for effective resident assessment (2-5).

The most useful system of evaluation is one that evaluators will be most apt to use (6), provided it offers an opportunity to deliver an assessment opportunity that is appropriate to the person being evaluated and sufficient detail so as to create a meaningful understanding of what has been observed without being excessively long and complex. Some evaluation types are useful in very specific settings. For example, an assessment of operative skills would be of no use in evaluating a resident's history-taking skills in the ambulatory office. An inventory or menu of evaluation types is needed to provide rich information on the ACGME competencies, and this can be comprised of any of a large number of established evaluation instruments. Accessing this when they are needed might be cumbersome as best, and impossible in the worst circumstances. Increasing faculty member efforts to complete evaluations would require a simple front-end experience to access a desired evaluation type, and rapid but invisible back-end processing of the entered information to make it available to both the learner and to the education leadership infrastructure. Faculty participation in resident evaluations will be greatly enhanced unnecessary workload is kept to a minimum. We sought to accomplish this with creating an automated evaluation selection and delivery system that would identify appropriate evaluations for residents in teaching cases and deliver them automatically to the corresponding teaching faculty.

### Materials and Methods

We built a secure, HIPAA-compliant, web-based platform for resident education management (7). The platform facilitated and tracked several aspects of resident education and performance, including case assignments, case logging, case outcomes, reading of targeted educational materials, and operative performance evaluations. The platform synced with operating room (OR) schedules and resident service rotation schedules to enable live case assignments and automatic matching of case details with evaluations. Based on the case procedure details and case staff, the platform identified relevant evaluations from a bank of available evaluations, including the Ottawa O-Score instrument rating of operative autonomy (8), Operative Performance Rating Systems (OPRS) evaluations (9), Entrustable Professional Activities (EPA) evaluations, Trauma non-operative evaluations, and resident self-evaluations. All evaluations were automatically paired with appropriate teaching cases and layered onto the operative schedule, where faculty and residents could easily find them and work them into their daily workflow. Faculty could choose whether to fill out one or more appropriate evaluations for each teaching case. For any teaching cases that still needed evaluations at the end of each day, the platform automatically sent brief reminder emails to the attendings to complete the evaluations, and upon completion it immediately pushed the evaluation results to the residents. Evaluation results were streamed into resident performance dashboards for residents, faculty, and program directors. The dashboards tracked resident learning with case experience, operative performance, and progress towards Accreditation Council for Graduate Medical Education (ACGME) requirements. The platform has been deployed multi-institutionally and across several departments.

For an initial test of the evaluation data quality, we measured the ability of the operative scores to stratify the residents by their program year (PGY) levels. Then, our principal measure of the usability of the platform's evaluation system was the time faculty spent to complete the evaluations. Each evaluation was structured as short set of Likert-scale questions, followed by optional comments. We split the evaluation responses into two sets, those with and those without comments, and on each set we measured the distribution of completion time using a Student's T-test with unequal variance, and linear models.

Delivering multiple appropriate evaluations together for the same cases afforded a unique opportunity to study resident performance across operative evaluations. We identified cases where faculty completed both O-Score and OPRS evaluations on the same resident. For these matching evaluations, we measured the Spearman rank-order correlation of the resident overall operative performance. We also investigated whether faculty completed both evaluations together in one sitting or at separate times. We measured the evaluation lag as the number of days between the teaching case and then submission of the corresponding evaluation. Finally, we explored correlations between pairs of questions across the evaluations.

### Results

1,230 O-Score evaluations, 106 OPRS evaluations, and 14 EPA evaluations were completed by 33 attendings for 67 residents from March 2017 to February 2019. Evaluations were completed quickly, with the completion time depending mostly on the level of detail that the attending chose to include in the optional comments. For evaluations without comments, the median completion times were 36±18 seconds for O-Score evaluations and 53±51 seconds for OPRS evaluations. For evaluations with comments, the times increased to 1.79±1.12 minutes for O-Score and 1.87±1.09 minutes for OPRS (t-test with unequal variance, p<0.00001). The overall evaluation completion time varied approximately linearly with comment length (r=0.85, p<0.00001 for O-Score, and r=0.54, p=0.001 for OPRS).

There were 74 teaching cases for which faculty completed both the O-Score and OPRS evaluation for the same resident, allowing for direct analysis of the timing and scoring across the paired evaluations. Faculty almost always completed both evaluations in one session, within a few days of the case (robust linear regression, r=0.97, p<0.0001) and within 1 minute±38 seconds of each other.

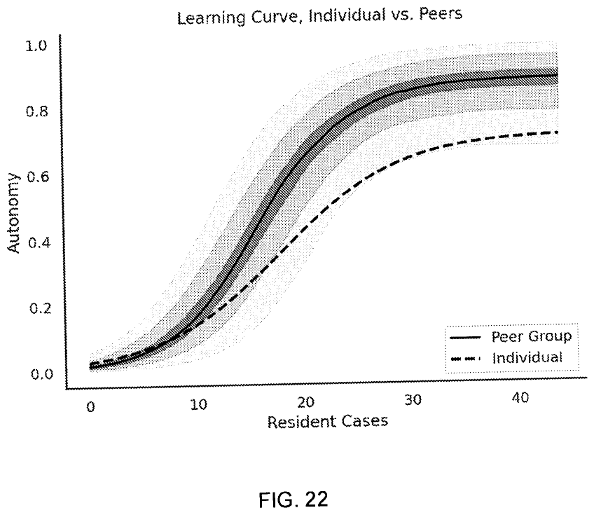

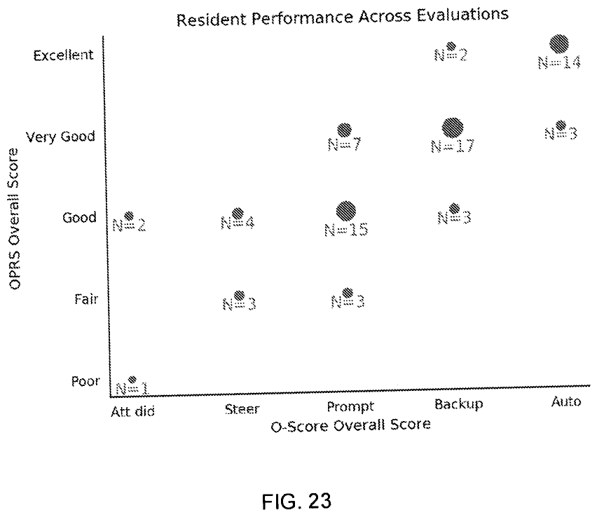

The paired evaluations showed high correlation for resident overall operative performance (Spearman's rho=0.84, p<0.00001) (FIG. 22). We measured the correlation across all pairs of questions across evaluations. The pairwise correlations were consistently high (rho>0.7) with the except of knot tying, which showed very little correlation across the other skills.

### Conclusions

The platform enabled flexibility in the evaluation process of resident operative performance. By integrating data from the department OR schedules, faculty staff profiles, resident profiles, and multiple types of evaluations, the platform automatically identified teaching cases and matched them with appropriate evaluations. By removing the friction from the evaluation selection and delivery process, it was much easier for time-pressed faculty to participate and complete their evaluations, even multiple evaluations per case. The platform improved the evaluation process for three relevant parties: 1) Faculty see appropriate evaluations in their personal operative schedules and get automated reminder emails, 2) Residents get much more timely feedback on their performance, and they don't have to do any set-up work to create the evaluations or send them to their attendings, and 3) Program directors experience much higher compliance rates from their teaching faculty and see their residents' performance trends in a real-time dashboard. One goal of the evaluation delivery system was to enable a virtuous feedback-and-learning cycle, where faculty would participate more, feeling that their evaluating time was valuable, as their feedback was delivered to the residents in real time soon after each case. And the residents would learn earlier how to improve their performance, and therefore demonstrate accelerated improvement throughout their service rotation with the faculty.

The proactive delivery and sub-minute completion times of the evaluations help explain their sustained use. The Likert-scale evaluations were short and quick enough for the faculty to fold into their daily workflow without much burden, and the evaluation comments allowed for additional feedback and guidance to the residents as needed. The paired evaluations demonstrated generally high correlations across their questions, indicating a well-balanced skills progression as residents gained operative experience. However, the notable outlier was knot tying, which showed no correlation to the other skills. Perhaps knot tying is a mechanical skill that is taught early in surgery residency and can be practiced in isolation, before the resident has the experience or background knowledge needed for higher-level skills, such as planning and decision-making in the OR, and general efficiency with time and motion during procedural steps. By comparing questions from several evaluation sources, it becomes possible to find an optimal set of predictive questions that minimize faculty burden and therefore maximize faculty participation, and maximize actionable utility to the residents (practitioners). Multi-evaluation data collected in a large scale can possibly reorient and accelerate the evaluation design process. Rather than carrying out a prolonged study to validate a fixed evaluation, a platform that continuously tracks faculty participation and resident performance improvement could enable a “rolling” strategy for prioritizing and selecting informative and actionable questions from several sources and packaging them into optimal, short evaluations delivered to the right faculty at the right time in their residents' (practitioners') educational journeys.

As a next step for the educational platform, these case-based evaluations can be combined and summarized into self-assembling consensus evaluations. The platform can present a coherent summary of all the recent evaluations to the evaluating faculty member, to facilitate the completion of end-of-rotation evaluations. The performance data could also be aggregated and structured according the ACGME milestones for program-level reporting. Currently, we are also helping faculty build their own custom procedure-specific evaluations, targeted at important procedural steps in common case types.

Referring to the figures, FIG. 9 shows how the platform integrates data from the OR schedule and assigned case staff, along with a data bank of available evaluations, to find appropriate evaluation and match them to each teaching case. The two evaluations stratified the residents across program year levels (p<0.0001). A larger average ORPS performance score for PGY 1 residents (practitioners) could have resulted from less complex cases appropriate for beginning surgery residents. Faculty completed the evaluations quickly, especially when they opted not to include the optional comments (p<0.00001). Most of the evaluation time was due to writing comments (p<0.0001). Faculty almost always completed both evaluations together, within a few days of performing the case with the resident (p<0.0001).

FIG. 22 shows the distribution for paired O-Score and OPRS evaluations showed high correlation (rho=0.84, p<0.00001) for resident (practitioner) overall operative performance. The size of the dots indicates the number of matching evaluations at each score level. Comparing questions across multiple matched evaluations enables a detailed view of the response patterns. In this subset, most questions demonstrated moderate correlation, with the exception of knot tying. Perhaps because knot tying is an early-level mechanical skill, it did not correlate with broader skills that require more experience and background knowledge.

### REFERENCES FOR EXAMPLE 2, ABOVE

1. Anderson P A. Giving feedback on clinical skills: are we starving our young? J Grad Med Educ. 2012; 4:154-8
2. Williams R G, Verhulst S, Colliver J A, Sanfey H, Chen X, Dunnington G L. A template for reliable assessment of resident operative performance: assessment intervals, numbers of cases and raters. Surgery. 2012 October; 152(4):517-24; discussion 524-7. doi: 10.1016/j.surg.2012.07.004. Epub 2012 Aug. 28.
3. Dougherty P, Kasten S J, Reynolds R K, Prince M E and Lypson, ML. Intraoperative assessment of residents. J Grad Med Educ. 2013 June; 5(2):333-4. doi: 10.4300/JGME-D-13-00074.1.
4. Williams R G, Swanson D B, Fryer J P, Meyerson S L, Bohnen J D, Dunnington G L, Scully R E, Schuller M C, George B C. How Many Observations are Needed to Assess a Surgical Trainee's State of Operative Competency? Ann Surg. 2019 February; 269(2):377-382. doi: 10.1097/SLA.0000000000002554.
5. Fryer J P, Teitelbaum E N, George B C, Schuller M C, Meyerson S L, Theodorou C M, Kang J, Yang A, Zhao L, DaRosa D A. Effect of Ongoing Assessment of Resident Operative Autonomy on the Operating Room Environment. J Surg Educ. 2018 March−April; 75(2):333-343. doi: 10.1016/j.jsurg.2016.11.018. Epub 2017 Mar. 28.
6. Williams R G, Kim M J, Dunnington G L. Practice Guidelines for Operative Performance Assessments. Ann Surg. 2016 December; 264(6):934-948.
7. Thanawala R, Jesneck J, Seymour N E. “Novel Educational Information Management Platform Improves the Surgical Skill Evaluation Process of Surgical Residents.” Journal of surgical education 75.6 (2018): e204-e211.
8. Gofton W T, Dudek N L, Wood T J, Balaa F, Hamstra S J. The Ottawa Surgical Competency Operating Room Evaluation (O-SCORE): a tool to assess surgical competence. Acad Med. 2012 October; 87(10):1401-7.
9. Larson J L, Williams R G, Ketchum J, Boehler M L, Dunnington G L. Feasibility, reliability and validity of an operative performance rating system for evaluating surgery residents. Surgery. 2005 October; 138(4):640-7; discussion 647-9.

### Example 3

Inferring Resident Autonomy for Surgical Procedures with Learning Curves

The American Board of Surgery expects residents to be proficient, safe, and autonomous across 132 “Core” surgical procedures in order to graduate and become practicing surgeons. For surgical educators, it can be a daunting task to solicit and assimilate performance feedback across a program's residents, especially in a timely, comprehensive, and quantitative manner. We propose a learning curve model that incorporates surgical case history along with Likert-scale and Zwisch-scale evaluation data to infer and quantify resident operative autonomy.

### Methods

We built a HIPAA-compliant web-based platform to track resident operative assignments and to link embedded evaluation instruments to procedure type. The platform delivered multiple evaluation types, including Ottawa O-Score autonomy evaluations. Autonomy scores were gathered across teaching faculty and combined with the residents' history of case assignments. For this analysis we focused on cholecystectomy cases. The data were entered into a logistic learning curve model, including estimates for the resident's learning lag (the number of cases needed until rapid learning), the maximum learning rate, and the autonomy limit (the maximum autonomy level we expect the resident to achieve after a large number of cases). The learning curve model included an ordinal response component, which inferred the resident's actual autonomy level from the faculty's ordinal Likert-scale ratings. It also inferred the faculty's implicit “hawk or dove” grader bias, while accounting for reported case complexity. The model was applied to each resident across the program, creating a learning baseline against which each individual resident can be compared to his or her peers.

### Results

129 evaluations for cholecystectomy cases were completed by 12 attendings for 31 residents over about 20 months. The learning curves for the residents clustered into an early-learning group of senior residents and a later-learning group of junior residents.

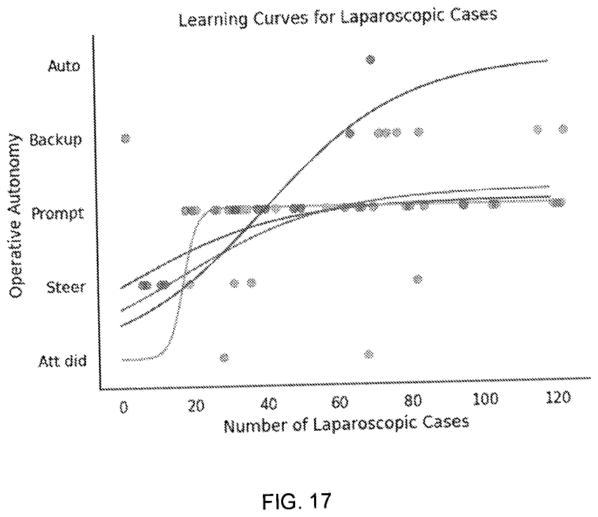

Referring to the figures, FIG. 17 shows learning curve distributions for pre-vs post-intervention. More specifically, this figure shows the learning curves for the residents, before the teaching intervention and after the intervention. By “intervention”, is meant that they were given extra training, support, and practice exercises by their teaching faculty, in order to help bring their operative performance up to an acceptable level. Here the learning bands show the distribution of the learning curves. The width of the bands shows the model confidence. The dense region in the middle is the most likely. As more evaluations are added, the model will have further data to work with and can iteratively produce more confident predictions, so the bands will likely converge. Also, it is seen that the teaching intervention was successful, because it shifted the residents' (practitioners') curve up, meaning that going forward they are likely to be more independent surgeons for laparoscopic cases.

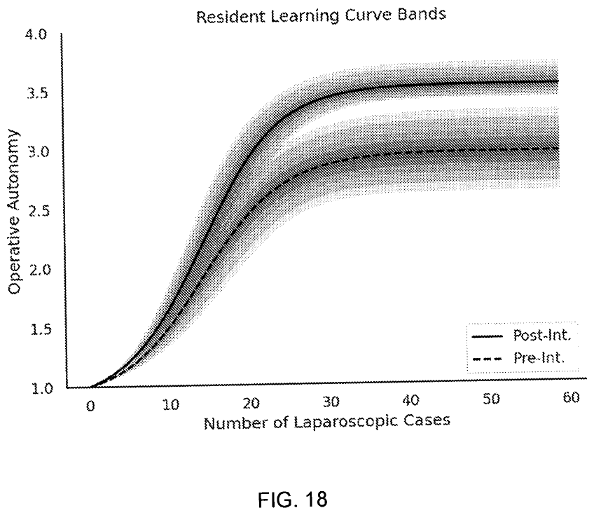

FIG. 18 is a plot showing posterior samples of the learning curves for a group of residents as a function of cases performed.

FIG. 22 shows a learning curve for an individual versus a composite of the learning curves for the peer group. The y-axis shows the level of autonomy rating, from lowest to highest: attending surgeon performed; steered; prompted; back-up, and auto as described further in Table 2.

### Example 4

### User Interface for Augmented Clinical Schedule:

This example row from a schedule shows a single case or patient encounter. With the case details are action buttons, for example for case logging, evaluating other case staff members, and editing the case details.

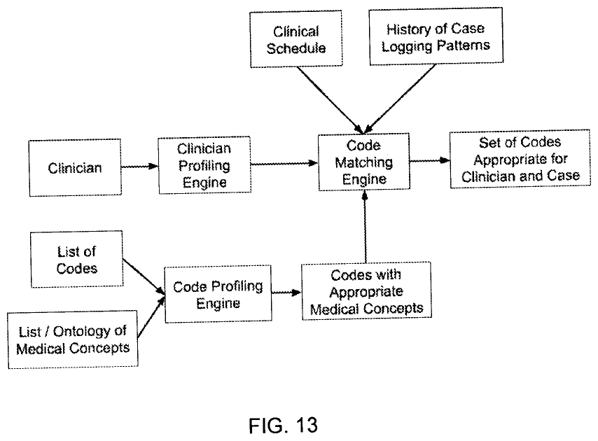

Matching Clinical Codes for Clinical Encounter Logging: The code matching system aggregates clinical data, clinical schedules, and historical case logging data to match appropriate codes for each clinician and clinical encounter. See. FIG. 13.

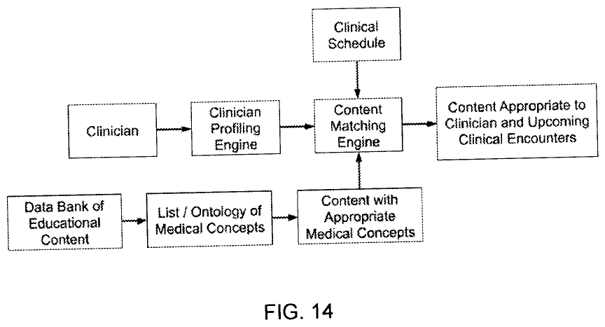

Matching Educational Content for Targeted Education: The Targeted Education System aggregates medical educational content, clinical schedules, and clinician practice patterns to match appropriate, high-quality educational content to each clinical for upcoming clinical encounters. See FIG. 14.

### Predicting Case Volume:

A) A medical learner's cumulative case volume over time. The volume is known from the start of his program until the present time, when statistical models are used to predict the case volume at a given future time, such as the learner's graduation date. The shaded area shows the credible band, and the center line shows the most likely value for the case volume. The horizontal dotted line shows the minimum number of cases required by the educational program, ACGME. See FIG. 24B.

B) The probability of achieving each number of cases at a given future time.

### Example 5

### Use of a Secure Web-Based Data Management Platform to Track Resident Operative Performance and Program Educational Quality Over Time

Objective: In surgery residency programs, ACGME mandated performance assessment can include assessment in the operating room to demonstrate that necessary quality and autonomy goals are achieved by the conclusion of training. For the past three years, our institution has used The Ottawa Surgical Competency Operating Room Evaluation (O-SCORE) instrument to assess and track operative skills. Evaluation is accomplished in near real-time using a secure web-based platform for data management and analytics (Firefly™). Simultaneous to access of the platform's case logging function, the O-SCORE instrument is delivered to faculty members for rapid completion, facilitating quality and timeliness of feedback. We sought to demonstrate the platform's utility in detecting operative performance changes over time in response to focused educational interventions based on stored case log and O-SCORE data.

Design: Resident performance for the most frequently performed laparoscopic procedures (cholecystectomy, appendectomy, inguinal hernia repair, ventral hernia repair) were examined over three successive academic years (2016-2019). During this time, 4 of 36 residents received program-assigned supplemental simulation training to improve laparoscopic skills. O-SCORE data for these residents were extracted from peer data, which were used for comparisons. Assigned training consisted of a range of videoscopic and virtual reality skills drills with performance objectives. O-SCORE response items were converted to integers and overall autonomy scores were compared before and after educational interventions (Student's t-tests). These scores were also compared to aggregate scores in the non-intervention group. Individual learning curves were used to characterize patterns of improvement over time.

Setting: Hospital Institutional Tertiary Care Center.

Participants: PGY2 through PGY4 general surgery residents (n=36).

Results: During the period of review, 3325 resident cases were identified meeting the case type criteria. As expected, overall autonomy increased with the number of cases performed. The four residents who had been assigned supplemental training (6-18 months) had pre-intervention score averages that were lower than that of the non-intervention group (2.25±0.43 vs 3.57±1.02; p<0.0001). During the respective intervention periods, all four residents improved autonomy scores (increase to 3.40±0.61; p<0.0001). Similar improvements were observed for tissue handling, instrument handling, bimanual dexterity, visuospatial skill, and operative efficiency component skills. Post-intervention scores were not significantly different compared to scores for the non-intervention group.

Conclusions: The Firefly™ platform proved to be very effective in tracking responses to supplemental training deemed important to close defined skills gaps in laparoscopic surgery. This could be seen both in individual and in aggregated data. We were gratified that at the conclusion of the supplemental training, O-SCORE results for the intervention group were not different than those seen in the non-intervention group.

Abbreviations: ACGME (Accreditation Council for Graduate Medical Education); O-SCORE (Ottawa Surgical Competency Operating Room Evaluation Score)

The skilled performance of surgery is extraordinarily demanding of practitioners at all levels of experience, and deficient surgeon skills are widely felt to negatively impact patient outcomes (1,2). Even with protections in place to limit duty hours, residency training in surgery continues to be arduous and lengthy with the overriding goal of preparing the trainee for safe, independent surgical practice. The process of training includes, by design, progressive withdrawal of direct supervision as experience, and commensurately, skills, knowledge, and confidence are gained. The Accreditation Council for Graduate Medical Education (ACGME) core program requirements for general surgery training (3) specify how this must occur in both general and specific terms. Educational tools that are expected to be used include access and exposure to core content, simulation, operative case experience under supervision (direct or indirect), and assessment methods that aim to both model and to make summative determinations about performance. Although all training programs seek to maximize residents' core competencies, the general means to accomplish this goal are not highly standardized, and in fact there is substantial “wiggle room” in designing curricula with substantial variations in nonclinical educational experiences.

Understanding each resident's areas of strength and weakness provides an opportunity to tailor training, including the use of simulation lab-based training, to the most applicable content needed to ensure efficient achievement of educational goals. The success of any such effort begins with the ability to identify the need for training and ends with demonstration that the desired performance has been attained. This requires effective assessment. Educational interventions that aim to address the training need(s) must also be available and utilized effectively in order to spur development. Effective assessment methods offer the opportunity to monitor performance on an individual basis and in groups of residents. With appropriate analytic capabilities, these performance data can provide a view of educational effectiveness at a programmatic level as well.

Having already shown that intelligent, technology-based operative assessment delivery along with incentivization of assessment completion (4) result in rapid availability of evaluations, we sought to determine if this established assessment model, when used with other evaluative data, could identify both the need for supplemental laparoscopic skills training and the collective effectiveness of our residency program's efforts to improve laparoscopic surgical performance based on the program's routine use these tools in the course of formative education.

### Materials and Methods

Ottawa Surgical Competency Operating Room Evaluation (O-SCORE): The O-SCORE (5,6) is an instrument to assess operative skills of individual residents on a case-by-case basis. This tool was introduced to the Hospital Surgery Residency: The O-SCORE, as described by its University of Ottawa developers, is a 9-item evaluative tool designed to assess technical competence with 8 items related to preprocedural planning, case preparation, knowledge of procedure steps, technical performance, visuospatial skills, postprocedural plan, efficiency and flow, communication, and overall performance. These are rated using a scale intended to reflect the degree of autonomy or independence demonstrated to the evaluator (Table 2). An additional item, answered “yes” or “no”, pertains to the residents' ability to do the case independently. In our implementation model, the form was expanded to 12 scaled items by specifying operative skills to include four separate items for evaluation of knot-tying, tissue handling, instrument handling, and ability to assist. Evaluations were delivered to faculty members using a secure web-based platform (Firefly Lab, Cambridge, MA) which matched the specific evaluation to the patient, proposed procedure, faculty member, and resident assigned to the case, using machine intelligence algorithms that also aided post-procedure case logging for both residents and faculty members (4). Evaluation and logging capabilities were optimized for use in web browser windows on both computers and hand-held devices. Firefly™ platform integrated analytics were used to obtain evaluative data over three successive academic years (2016-2019) for the four most frequently performed laparoscopic general surgery procedures: cholecystectomy, appendectomy, inguinal hernia repair, and ventral hernia repair. Integers on the autonomy scale ranged from 1 to 5, corresponding to attending “I had to do” continuing up to “I did not need to be there” representing maximum resident autonomy for all assessment items (Table 2). To make these descriptors more display-friendly on cell phone screens, they were shortened to terms such as “I did” and “Auto”.

Training interventions: During the reviewed period, four of 36 residents, postgraduate year 2-4, were assigned individual learning plans consisting of supplemental simulation training with the aim of improving laparoscopic skills. The determination of need to receive this training was based on a combination of evaluative information sets that included O-SCORE results, end-of-rotation evaluations, and ad hoc commentary received by the Surgical Education Office. This determination was a subjective one made by the Surgical Education Committee and prompted preparation of individual learning plans that required at least weekly 1-hour sessions in the Hospital Simulation Center-Goldberg Surgical Skills Lab, beyond the normal weekly 1-hour simulation selectives assignments. Supplemental training consisted of a range of videoscopic and virtual reality skills drills with clear performance objectives and lab-based coaching for 30-52 weeks. During the period over which this training occurred, residents exercised their usual clinical responsibilities, including operative experiences.

O-SCORE data for these four residents were extracted from the peer data for other residents, which were used as a control dataset for comparison purposes. Numerical O-SCORE individual skills deemed relevant to their lab-based training as well as overall scores were analyzed. Numerical data are expressed as mean±standard error (or 95% confidence intervals for graphed data), and compared before and after supplemental educational interventions (paired Student's t-tests). These scores were also compared to aggregate scores in the non-intervention group (unpaired Student's t-tests). Grouped learning curves were modeled from longitudinal assessments and logged case numbers for individual residents. Our methodology enables the calculation of the most likely learning curve for each resident group. By fitting the curve to the observed evaluation scores, it calculates the most likely values for the residents' learning rates and predicted maximum autonomy levels. We used a generalized logistic curve under a statistical framework to compensate for the reality of fewer assessments than logged relevant cases. This model fitted curves to assessment data and inferred curve shape using Markov chain Monte Carlo sampling (7), and using the No-U-Turn Sampler (8) for computationally efficient sampling of the curve parameters for each group. The evaluation ordinal ratings were used to infer each resident's operative autonomy level, learning rate, and predicted maximum autonomy level. The model also inferred and accounted for case complexity and each teaching faculty member's “hawk vs dove” rater bias (9).

This retrospective analysis of resident performance was reviewed by the Hospital Institutional Review Board as a quality assurance activity and was deemed not to constitute human subjects research.

### Results

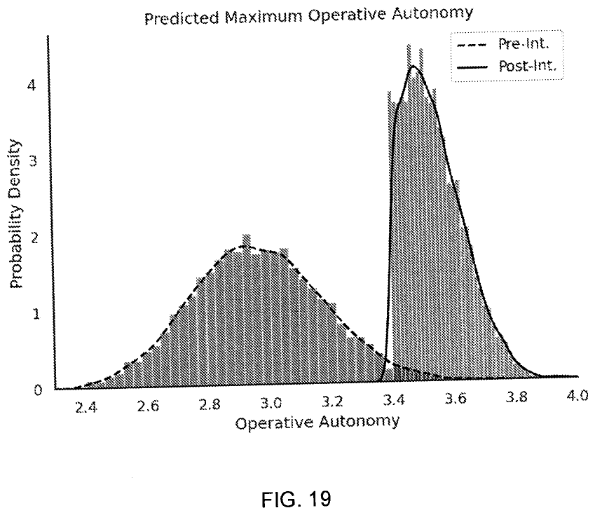

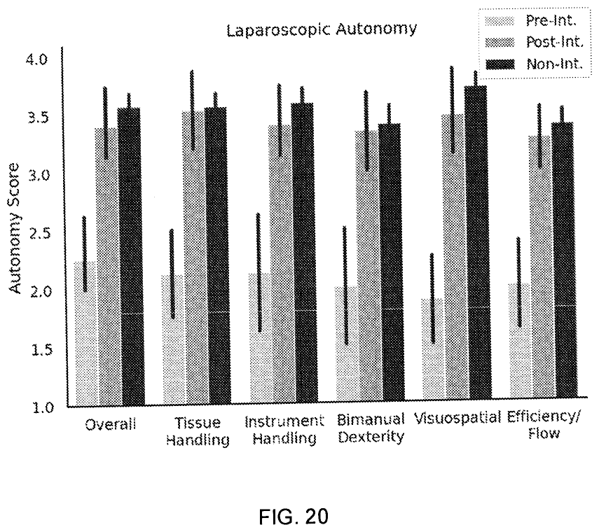

During the period of review, 3325 logged resident cases and 369 O-SCORE assessments were identified as meeting the case type criteria. From these, 54 assessments were available for residents in the educational intervention group. As expected, modeled learning curves expressing interpolated performance showed that all residents improve as the number of cases performed increases. However, for residents determined to need supplemental training, the pre-intervention curve shows a clear pattern of performance lag relative to the non-intervention curve (FIG. 18), with suggestion of a blunted rate of improvement and a lower level of operative autonomy at the projected point of slowed rate of improvement (25-30 cases) compared to both the post- and non-intervention curves. Examining post-intervention performance, this performance deficit had improved substantially with the learning rate and the predicted maximum autonomy levels more closely resembling those of the non-intervention group (FIG. 18). Further, histogram analysis shows the posterior predictive value of the maximum autonomy level increased significantly from the pre-intervention to the post-intervention (p<0.0001, FIG. 19). However, these did not approach the much higher posterior predictive value of the nonintervention group which was based on a much larger number of observations.

Examination of mean performance scores demonstrates a similar pattern of performance difference between pre- and post-intervention results for residents in the educational intervention group, and between this group and the non-intervention group (Table 3). During the six-month period prior to assignment of supplemental training, the four residents in the intervention group were noted to have average scores that were significantly lower than the non-intervention group averages (“overall” scores 2.25±0.43 vs 3.57±1.02 respectively; p<0.0001). Over the course of their respective intervention periods, all four residents improved O-SCORE results (increase in “overall” scores from 2.25±0.43 to 3.40±0.61; p<0.0001). In addition to overall autonomy, similarly significant improvements were observed for tissue handling, instrument handling, bimanual dexterity, and visuospatial component skills (FIG. 19). Although not necessarily the focus of lab-based training, “Efficiency and Flow” results showed similar improvements. During the post-intervention period for the educational intervention group, these component and overall scores were not significantly different from corresponding scores for the non-intervention group (Table 3).

### Conclusions

In recent years, the surgical residency experience has changed in fundamental ways for trainees and faculty members, raising questions of whether the requisite skill levels for the independent practice of surgery can reliably be achieved by chief residents after completion of five years of clinical training. The cited factors that might impede this goal include limitations on hours worked (10), decreased exposure to a sufficiently large number and broad range of operations (11-13), and barriers to the offer of opportunities to exercise a high degree of independence during operative cases (14). Probably no less important, momentous changes in surgical methods and tools have undoubtedly added a layer of complexity to training that might impact opportunities for resident operative autonomy. Without strong mitigation steps, perhaps unsurprisingly, graduating residents might feel underprepared for independent practice or, as in one report, seem to underperform in the view of fellowship program directors when first confronted with common operative responsibilities (15,16). While it is difficult to draw broad conclusions from these observations without compelling data to suggest that patient outcomes are adversely affected, efforts have been made in the past few years to facilitate higher degrees of independent resident practice. This is especially important to achieve while the strong supervisory infrastructure associated with residency is available.

Among the ways improved resident preparation might be accomplished include increasing the number and range of opportunities to exercise independent practice safely (17). Active learning techniques improve knowledge acquisition and retention for surgical learners (18), and abundant experience shows that simulation methods can amplify surgical ability by providing practice of component skills and procedures outside of the operating room (19-21). It is widely accepted that use of simulation can increase trainee operative skills, and some limited data show that simulation training can improve selected patient outcomes (22). However, few studies have specifically examined the effects of simulation training on surgeon confidence or operative autonomy, and those that do are limited in sample size and scope (23,24). In addition, there is surprisingly little published experience with lab-based training as a tool to tackle low assessed levels of technical performance in the OR. Gas, et al. reported that when performance is assessed carefully and remedial simulation is applied systematically with clearly-defined goals, performance shortfalls on skills station tasks can be corrected (25). The authors made use of the terms “poor performance” and “remediation” to describe trainee characteristics and actions taken, but these did not have a measured clinical context. In truth, the effectiveness of formal remediation of knowledge, behavior, or skills in surgical residency is not well studied. Similarly, the relationship of the need for remediation to attrition is also not well established, although we maintain that some performance characteristics may not be remediable and should appropriately result in residency program attrition. In a survey-based study of categorical residents in 21 American surgery residencies, Schwed, et al. reported that use of effective remediation correlated with lower attrition rates (26). Speculating on the reasons for this observation, the authors suggested that programs using remediation may take greater ownership of performance deficits and take greater pains to help residents correct performance deficits.

The belief that at least some low technical performance characteristics in trainees can be effectively addressed with education tools including lab-based simulation practice was one of the benefits we had hoped to achieve by building our Firefly™-based assessment system. The current results show that with the use of a dynamic and widely-implemented framework of operative skills assessment and active modeling of lab-based training experiences, operative skill and autonomy can be improved after having been defined as insufficient. In this case, programmatic recognition of the need for focused development did not necessarily require defining a need for “remediation.” There are expected variations in observed skills during the course of residency training (12). Without clear evidence of how these variations impact clinical outcomes, professional standing, or other career difficulties, assigning these descriptors implies a level of significance that might not have a consistent basis. That is not to say that the terms are not applicable or that targeted skills development is not of critical importance. There is, however, a need to frame the goals of such skills development around the evidence that it is of value, and that they contribute to clinical performance improvement. In our own program we have, somewhat arbitrarily, defined the need of “remediation” in a very formal sense to describe a state of escalation of concern about failing performance. By consensus, our institution's residency programs have further reserved the term for situations where success of corrective measures is truly in question and non-promotion or employment action may be justified. Labels or measures that stigmatize can negatively affect responsiveness to efforts to achieve improvement (27). In some settings, such labels have implications such as reportability to regulatory bodies, and can have further implications to future licensure or credentialing. None of the residents for whom data are reported here were identified as “failing” and the subjective observations made about the observed skills were generally in the context of expected level-appropriate skills. None of the learning plans were presented to participating residents as “remediation.” The learning plans were formalized, however, with specific requirements, the most important of which was the message that supplemental training was mandatory and compliance would be monitored. In all instances, supplemental training occurred over a period of months and, in some situations, residents had to be reminded to resume sessions after missed sessions were reported by the Simulation Center staff.

Although the retrospectively aggregated data we report showed a temporary, correctable performance lag for selected residents, there are important limitations that must be noted that make it difficult to make detailed characterizations of performance changes or to comment on causation in regard to the educational interventions. Sweeping statements based on performance patterns for only four trainees are clearly unwarranted. The amount, frequency, and precise makeup of the supplemental training were not consistently recorded. The actual number of sessions, hours in training, and specific lab goals achieved for each resident were only known in general terms and there was no systematic accounting of self-directed practice sessions. There is also no information available on other opportunities for learning or other educational actions taken either in or out of the operating room that may have affected the longitudinal results of individual O-SCORE data. Although operative autonomy and operative skill are not synonymous, a recent collaborative examination of attending decision-making on awarding resident autonomy in the OR suggested that the most important determinant is residents' perceived performance (28). The scaled items used in the O-SCORE instrument infer level of competency based on the perceived need of the evaluator to do portions (or all) of the case. For results to be consistent between evaluators, a resident's opportunity to exercise autonomy would have to be granted on a fairly uniform basis. Despite sophisticated embedded mathematical tools in the Firefly™ platform to discern whether this occurs, varying thresholds for intervention between evaluators who, as surgeons of record, are in the position to skew O-SCORE results by their own biases, were difficult to control for in an analysis of this limited size. This can be studied further, however, by looking for patterns of “hawk” and “dove” grading behaviors that might be evident in larger data sets.

Our study strengths included wide use of a fairly well-studied assessment instrument, albeit in somewhat modified form. The O-SCORE tool has been shown to produce accurate and reproducible results in the evaluation of surgical competence in trainees, both in the operating room and in simulation (4,5). Our recent experience with use of Firefly™-facilitated modified O-SCORE assessments showed that evaluations were completed and pushed electronically to the assessed residents rapidly, the process completed in the majority of cases within a few hours (6). However, even when available for use for the totality of resident cases performed, only 11% of the resident-performed laparoscopic cases received O-SCORE assessments during the 2016-19 review period. These were completed by a core group of trained evaluators all of whom were highly experienced full-time faculty minimally invasive surgeons. In the course of the program's formative education efforts, our hope was that these assessments contributed in a meaningful way to residents' performance feedback and provided a basis for performance improvement, in addition to keeping residency program leadership informed of performance issues. However, the success of lab-based interventions to help trainees add skills defined as lagging but necessary to clinical development is not a given. We would like to gain a greater degree of confidence that close tracking of resident assessment results provides a meaningful basis to model training and intervene early to ensure success in training efforts.

Until the current report, we had not used assessment data as a means of tracking the effectiveness of specific educational measures employed by the residency program. The use of the Firefly™ platform to comprehensively manage evaluative information enabled us to query and analyze grouped and individual data in order to address an educational quality question that would have been more cumbersome to answer without the availability of the platform. Other web-based systems for delivery of assessments and compilation of assessment data have been used successfully (29-31). All are examples of the application of technology to the problem of ensuring high quality evaluations and to the logistical problem of facilitating timely and frequent completion. However, the analysis of compiled performance data to ensure that larger program actions are helping to maintain the quality of education has not been a major focus of these efforts. We found that the integration of analytic tools into the same platform used for evaluation management is critical to monitoring the overall quality of educational processes. It is now standard practice for our team to not only examine individual resident progress with increasingly frequent use of these tools, but to also examine grouped data with the Firefly™ platform's analytic tools in order to determine if additions and changes in our educational program impact residents' clinical abilities.

TABLE 3

| | | | | Pre- vs Post- | Pre- vs Non- | Post- vs Non- |
|---|---|---|---|---|---|---|
| Metric | Pre-Int. | Post-Int. | Non-Int. | Int p-value | Int p-value | Int p-value |
| Overall | 2.25 ± 0.43 | 3.40 ± 0.61 | 3.57 ± 1.02 | 0.000037 | 0.000089 | 0.35 |
| Tissue Handling | 2.12 ± 0.60 | 3.53 ± 0.72 | 3.56 ± 1.09 | 0.00028 | 0.00021 | 0.91 |
| Instrument Handling | 2.12 ± 0.78 | 3.40 ± 0.61 | 3.59 ± 1.1 | 0.0015 | 0.0029 | 0.30 |
| Bimanual Dexterity | 2.0 ± 0.71 | 3.33 ± 0.70 | 3.4 ± 1.32 | 0.00097 | 0.0011 | 0.76 |
| Visuospatial | 1.88 ± 0.6 | 3.47 ± 0.72 | 3.7 ± 1.13 | 0.000048 | 0.000058 | 0.26 |
| Efficiency/Flow | 2.0 ± 0.50 | 3.27 ± 0.57 | 3.38 ± 1.11 | 0.000085 | 0.000090 | 0.51 |

Table 3 Abbreviations:
Pre-Int. = pre-intervention;
Post-Int. = post-intervention;
Non-Int. = non-intervention

### REFERENCES FOR EXAMPLE 5 ABOVE

1. Birkmeyer J D, Finks J F, O'Reilly A, Oerline M, Carlin A M, Nunn A R, Dimick J, Banerjee M, Birkmeyer N J; Michigan Bariatric Surgery Collaborative. Surgical skill and complication rates after bariatric surgery. N Engl J Med. 2013; 369(15):1434-42. https//doi: 10.1056/NEJMsa1300625.
2. Abid M A, Li Y W, Cummings C W, Bhatti N I. Patient outcomes as a measure of surgical technical skills: Does surgical competency matter? A systematic review. Otorinolaringologia. 2016; 66(4):99-106.
3. ACGME Program Requirements for Graduate Medical Education in General Surgery. https://www.acgme.org/Portals/0/PFAssets/ProgramRequirements/440_GeneralSurg ery_2019. pdf?ver=2019-06-19-092818-273
4. Thanawala R, Jesneck J, Seymour N. Novel Educational Information Management Platform Improves the Surgical Skill Evaluation Process of Surgical Residents. J Surg Education. 2018; 75(6):e204-e211. https//doi: 10.1016/j.jsurg.2018.06.004.
5. MacEwan M J, Dudek N L, Wood T J, Gofton W T. Continued validation of the O-SCORE (Ottawa Surgical Competency Operating Room Evaluation): use in the simulated environment. Teach Learn Med. 2016; 28(1):72-9. https//doi: 10.1080/10401334.2015.1107483.
6. Gofton W T, Dudek, NL, Wood T J, Balaa F, Hamstra S J. The Ottawa Surgical Competency Operating Room Evaluation (O-SCORE): a tool to assess clinical competence. Acad Med. 2012; 87(10):1401-7. https//doi:10.5555/2627435.2638586.
7. Berg B A. Markov Chain Monte Carlo Simulations and Their Statistical Analysis. Singapore. World Scientific Publishing Co. Pte. Ltd., 2004.
8. Hoffman M D, Gelman A. The No-U-Turn sampler: adaptively setting path lengths in Hamiltonian Monte Carlo. J Mach Learn Res. 2014; 15(1): 1593-1623.
9. McManus I C, Thompson M, Mollon J. Assessment of examiner leniency and stringency (‘hawk-dove effect’) in the MRCP (UK) clinical examination (PACES) using multi-facet Rasch modelling. BMC Med Educ. 2006 Aug. 18; 6:42. https//doi.org/10.1186/1472-6920-6-42.
10. Ahmed N, Devitt K S, Keshet I, Spicer J, Imrie K, Feldman L, Cools-Lartigue J, Kayssi A, Lipsman N, Elmi M, Kulkarni A V, Parshuram C, Mainprize T, Warren R J, Fata P, Gorman M S, Feinberg S, Rutka J. A systematic review of the effects of resident duty hour restrictions in surgery: impact on resident wellness, training, and patient outcomes. Ann Surg. 2014; 259(6):1041-53. https//doi: 10.1097/SLA.0000000000000595.
11. Drake F T, Horvath K D, Goldin A B, Gow K W. The general surgery chief resident operative experience: 23 years of national ACGME case logs. JAMA Surg. 2013; 148(9):841-7. https//doi: 10.1001/jamasurg.2013.2919.
12. Quillin R C 3rd, Cortez A R, Pritts T A, Hanseman D J, Edwards M J, Davis B R. Operative variability among residents has increased since implementation of the 80-hour workweek. J Am Coll Surg. 2016; 222(6):1201-10. doi: 10.1016/j.jamcollsurg.2016.03.004. Epub 2016 Mar. 18.
13. Bell R H Jr. Why Johnny cannot operate. Surgery. 2009; 146(4):533-42. https//doi: 10.1016/j.surg.2009.06.044.
14. Hashimoto D A, Bynum W E 4th, Lillemoe K D, Sachdeva A K. See More, Do More, Teach More: Surgical Resident Autonomy and the Transition to Independent Practice. Acad Med. 2016; 91(6):757-60. https//doi: 10.1097/ACM.0000000000001142.
15. Bucholz E M, Sue G R, Yeo H, Roman S A, Bell R H Jr, Sosa J A. Our trainees' confidence results from a national survey of 4136 US general surgery residents. Arch Surg. 2011; 146(8):907-914. https//doi:10.1001/archsurg.2011.178
16. Mattar S G, Alseidi A A, Jones D B, Jeyarajah D R, Swanstrom L L, Aye R W, Wexner S D, Martinez J M, Ross S B, Awad M M, Franklin M E, Arregui M E, Schirmer B D, Minter R M. General surgery residency inadequately prepares trainees for fellowship: results of a survey of fellowship program directors. Ann Surg. 2013; 258(3):440-9. https//doi: 10.1097/SLA.0b013e3182a191ca.
17. Jarman B T, O'Heron C T, Kallies K J, Cogbill T H. Enhancing Confidence in Graduating General Surgery Residents: Establishing a Chief Surgery Resident Service at an Independent Academic Medical Center. J Surg Educ. 2018; 75(4):888-894. https//doi: 10.1016/j.jsurg.2017.12.012. Epub 2018 Feb. 3.
18. Luc J G Y, Antonoff M B. Active Learning in Medical Education: Application to the Training of Surgeons. J Med Educ Curric Dev. 2016 May 4; 3. pii: JMECD.S18929. doi: 10.4137/JMECD.S18929. eCollection 2016 January-December
19. Nagendran M, Gurusamy K S, Aggarwal R, Loizidou M, Davidson B R. Virtual reality training for surgical trainees in laparoscopic surgery. Cochrane Database Syst Rev. 2013; (8):CD006575. https//doi: 10.1002/14651858.CD006575.pub3.
20. Papanikolaou I G, Haidopoulos D, Paschopoulos M, Chatzipapas I, Loutradis D, Vlahos N F. Changing the way we train surgeons in the 21th century: A narrative comparative review focused on box trainers and virtual reality simulators. Eur J Obstet Gynecol Reprod Biol. 2019; 235:13-18. https//doi: 10.1016/j.ejogrb.2019.01.016.
21. Hanks J B. Simulation in Surgical Education: Influences of and Opportunities for the Southern Surgical Association. J Am Coll Surg. 2019; 228(4):317-328. https//doi: 10.1016/j.jamcollsurg.2018.12.029. Epub 2019 Jan. 17.
22. Cox T, Seymour N, Stefanidis D. Moving the Needle: Simulation's Impact on Patient Outcomes. Surg Clin North Am. 2015; 95(4):827-38. https//doi: 10.1016/j.suc.2015.03.005.
23. Kim S C, Fisher J G, Delman K A, Hinman J M, Srinivasan J K. Cadaver-Based Simulation Increases Resident Confidence, Initial Exposure to Fundamental Techniques, and May Augment Operative Autonomy. J Surg Educ. 2016; 73(6): e33-e41. https//doi: 10.1016/j.jsurg.2016.06.014.
24. Lesch H, Johnson E, Peters J, Cendan J C. VR simulation leads to enhanced procedural confidence in surgical trainees. J Surg Educ. 2020; 77(1):213-218. https//doi: 10.1016/j.jsurg.2019.08.008.
25. Gas B L, Buckarma E H, Mohan M, Pandian T K, Farley D R. Objective Assessment of General Surgery Residents Followed by Remediation. J Surg Educ. 2016; 73(6):e71-e76. https//doi: 10.1016/j.jsurg.2016.07.002.
26. Schwed A C, Lee S L, Salcedo E S, et al. Association of General Surgery Resident Remediation and Program Director Attitudes With Resident Attrition. JAMA Surg. 2017; 152(12):1134-1140. https//doi:10.1001/jamasurg.2017.2656
27. Kalet A, Chou C L, Ellaway R H. To fail is human: remediating remediation in medical education. Perspect Med Educ. 2017; 6(6):418-424. https//doi: 10.1007/s40037-017-0385-6.
28. Williams R G, George B C, Meyerson S L, Bohnen J D, Dunnington G L, Schuller M C, Torbeck L, Mullen J T, Auyang E, Chipman J G, Choi J, Choti M, Endean E, Foley E F, Mandell S, Meier A, Smink D S, Terhune K P, Wise P, DaRosa D, Soper N, Zwischenberger J B, Lillemoe K D, Fryer J P; Procedural Learning and Safety Collaborative. What factors influence attending surgeon decisions about resident autonomy in the operating room? Surgery. 2017; 162(6):1314-1319. https//doi: 10.1016/j.surg.2017.07.028.
29. Wohaibi E M, Earle D B, Ansanitis F E, Wait R B, Fernandez G, Seymour N E. A new web-based operative skills assessment tool effectively tracks progression in surgical resident performance. J Surg Educ. 2007; 64(6):333-41.
30. Wagner J P, Chen D C2, Donahue T R, Quach C, Hines O J, Hiatt J R, Tillou A. Assessment of resident operative performance using a real-time mobile Web system: preparing for the milestone age. J Surg Educ. 2014; 71(6):e41-6. https//doi: 10.1016/j.jsurg.2014.06.008.
31. Bohnen J D, George B C, Williams R G, Schuller M C, DaRosa D A, Torbeck L, Mullen J T, Meyerson S L, Auyang E D, Chipman J G, Choi J N, Choti M A, Endean E D, Foley E F, Mandell S P, Meier A H, Smink D S, Terhune K P, Wise P E, Soper N J, Zwischenberger J B, Lillemoe K D, Dunnington G L, Fryer J P; Procedural Learning and Safety Collaborative (PLSC). The Feasibility of Real-Time Intraoperative Performance Assessment With SIMPL (System for Improving and Measuring Procedural Learning): Early Experience From a Multi-institutional Trial. J Surg Educ. 2016; 73(6): e118-e130. https//doi: 10.1016/j.jsurg.2016.08.010.
32. Gundle K R, Mickelson D T, Cherones A, Black J, Hanel D P. Rapid Web-Based Platform for Assessment of Orthopedic Surgery Patient Care Milestones: A 2-Year Validation. J Surg Educ. 2017; 74(6):1116-1123. https//doi: 10.1016/j.jsurg.2017.05.001.
33. Van Heest A E, Agel J, Ames S E, Asghar F A, Harrast J J, Marsh J L, Patt J C, Sterling R S, Peabody T D. Resident Surgical Skills Web-Based Evaluation: A Comparison of 2 Assessment Tools. J Bone Joint Surg Am. 2019 Mar. 6; 101(5):e18. https//doi: 10.2106/JBJS.17.01512.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

At certain points throughout some of the Examples of the specification, some references are referred to using a number in parentheses. Those numbers correspond to the references listed at the end of that particular example. Other references are cited within other parts of the specification and other references are cited separately.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps of the methods or components of the compositions, it is also contemplated that the methods and compositions consist essentially of, or consist of, the recited steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

What is claimed is:

1. A data management platform for determining a competency score for a target healthcare professional, the platform comprising: a computer, a server or data storage system, a user interface, a non-transitory computer-readable medium storing computer program instructions, software for analyzing input data and providing an output, and a data array,
wherein the platform is configured to perform steps comprising:
acquiring clinical schedules indicating clinical procedures to be performed;
listing component tasks and required skills for each procedure;
assessing task complexity;
collecting performance evaluations for the target healthcare professional and a matched peer group of the target healthcare professional, for the performance of one or more selected procedures, each procedure having one or more tasks and an assigned clinical complexity value for the procedure and the one or more tasks thereof;
compiling the evaluations versus predetermined standards for the successful completion of each task and one or more steps thereof to provide performance parameters;
performing a computation to produce learning curves from the performance parameters for the target healthcare professional and the matched peer group of the target healthcare professional, wherein the computation is selected from the group consisting of statistical modeling, deep learning modeling, and machine learning modeling;
from the learning curves for the target healthcare professional, calculating a competency score for the target healthcare professional for the procedure and each task thereof; and
comparing the learning curves and skill levels for the procedure and each task thereof for the target healthcare professional to that of the matched peer group of the target healthcare professional to determine a competency score for the target healthcare professional.

2. The platform according to claim 1 wherein the computation to produce learning curves is a deep learning curve modeling comprising the step of performing a statistical sampling method calculation to produce one or more learning curves for the target healthcare professional and the matched peer group of the target healthcare professional.

3. The platform according to claim 1 wherein the healthcare professional is selected from the group consisting of medical students, interns, residents, fellows, doctors, physician assistants, nurses, nurses' aides, and medical technicians.

4. The platform according to claim 1 involving a teaching situation involving an evaluator healthcare professional and a target healthcare professional.

5. The platform according to claim 1, wherein the user interface is a graphical user interface configured to augment a clinical schedule with case-based actions; the graphical user interface comprising:
a first element showing a staff assignment for a clinical encounter; and
a second element juxtaposed to the first element and showing a button, a tag, a status label, or an actionable link for an encounter-related activity, such as case logging, performance evaluation, data quality control, and accessing medical educational content.

6. The platform according to claim 1 wherein the performance evaluations are provided manually.

7. The platform according to claim 1 that is Health Insurance Portability and Accountability Act compliant.

8. The platform according to claim 1, the platform further configured to comprise a step of determining a risk score, wherein the risk score indicates a probability of a clinical event achieving a predetermined patient outcome.

9. A method for determining a competency score for a target healthcare professional comprising the following steps:
acquiring clinical schedules indicating clinical procedures to be performed;
listing component tasks and required skills for each procedure;
assessing task complexity;
collecting performance evaluations for a target healthcare professional and a matched peer group of the target healthcare professional, for the performance of one or more selected procedures, each procedure having one or more tasks and an assigned clinical complexity value for the procedure and the one or more tasks thereof;
compiling the evaluations versus predetermined standards for the successful completion of each task and one or more steps thereof to provide performance parameters;
performing a computation to produce learning curves from the performance parameters for the target healthcare professional and the matched peer group of the target healthcare professional, wherein the computation is selected from the group consisting of statistical modeling, deep learning modeling, and machine learning modeling;
from the learning curves for the target healthcare professional, calculating a competency score for the target healthcare professional for the procedure and each task thereof; and
comparing the learning curves and skill levels for the procedure and each task thereof for the target healthcare professional to that of the matched peer group of the target healthcare professional to determine a competency score for the target healthcare professional.

10. The method according to claim 9 wherein the computation to produce learning curves is a deep learning curve modeling comprising the step of performing a statistical sampling method calculation to produce one or more learning curves for the target healthcare professional and the matched peer group of the target healthcare professional.

11. The method according to claim 9 wherein the healthcare professional is selected from the group consisting of medical students, interns, residents, fellows, doctors, physician assistants, nurses, nurses' aides, and medical technicians.

12. The method according to claim 9 involving a teaching situation involving an evaluator healthcare professional and a target healthcare professional.

13. The method according to claim 9, wherein the user interface is a graphical user interface configured to augment a clinical schedule with case-based actions; the graphical user interface comprising:
a first element showing a staff assignment for a clinical encounter; and
a second element juxtaposed to the first element and showing a button, a tag, a status label, or an actionable link for an encounter-related activity, such as case logging, performance evaluation, data quality control, and accessing medical educational content.

14. The method according to claim 9 wherein the performance evaluations are provided manually.

15. The method according to claim 9 wherein the platform is embedded in a health record data system in a hospital.

16. The method according to claim 9 that is Health Insurance Portability and Accountability Act compliant.

17. The method according to claim 9, further comprising determining a risk score, wherein the risk score indicates a probability of a clinical event achieving a predetermined patient outcome.

18. The method according to claim 17, wherein the risk score is calculated for an individual practitioner to perform a specific procedure.

19. The method according to claim 17, further comprising determining a multi-task aggregate competency score based on individual task scores for an overall procedure.

20. A data management platform for determining a competency score for a target professional, the platform comprising: a computer, a server or data storage system, a user interface, a non-transitory computer-readable medium storing computer program instructions, software for analyzing input data and providing an output, and a data array,
wherein the platform is configured to perform steps comprising:
acquiring schedules indicating activities to be performed;
listing component tasks and required skills for each activity;
assessing task complexity;
collecting performance evaluations for the target professional and a matched peer group of the target professional, for the performance of one or more selected activities, each activity having one or more tasks and an assigned complexity value for the activity and the one or more tasks thereof;
compiling the evaluations versus predetermined standards for the successful completion of each task and one or more steps thereof to provide performance parameters;
performing a computation to produce learning curves from the performance parameters for the target professional and the matched peer group of the target professional, wherein the computation is selected from the group consisting of statistical modeling, deep learning modeling, and machine learning modeling;
from the learning curves for the target professional, calculating a competency score for the target professional for the activity and each task thereof; and
comparing the learning curves and skill levels for the activity and each task thereof for the target professional to that of the matched peer group of the target professional to determine a competency score for the target professional.
